(19) United States
(12) Patent Application Publication
(10) Pub. No.: US 2026/0077174 A1
(43) Pub. Date: Mar. 19, 2026

(54) BALLOON MEDICAL SYSTEM

(71) Applicant: Medtronic Ireland Manufacturing Unlimited Company, Dublin (IE)

(72) Inventors: Brian J. Kelly, Oranmore (IE); Daniel M. King, Galway (IE); Eamonn A. Walsh, Galway (IE); David J. Hobbins, Galway (IE); Shane Nolan, Galway (IE); Tomas K. Kelly, Galway (IE)

(21) Appl. No.: 19/106,038

(22) PCT Filed: Aug. 28, 2023
(86) PCT No.: PCT/EP2023/073515

§ 371 Date: Feb. 24, 2025

Related U.S. Application Data
(60) Provisional application No. 63/374,046, filed on Aug. 31, 2022.

(51) Int. Cl. A61M 39/22; A61B 18/00; A61B 18/04

(52) U.S. Cl. CPC A61M 39/227; A61B 18/04; A61B 2018/00404; A61B 2018/00511; A61B 2018/00577; A61B 2018/046

(57) ABSTRACT

A medical system is configured to deliver a therapeutic agent to a patient using a catheter system having a balloon. A valve manifold includes a first valve configured to transition to a first valve open position based on an inflation pressure of the balloon. The valve manifold includes a second valve configured to position in a second valve open using an actuator. The valve manifold is configured to fluidically couple an inlet flow path and the outlet flow path to pressurize of the therapeutic agent when the first valve is in the first valve open position and the second valve is in the second valve open position. The valve manifold is configured to fluidically isolate the inlet flow path and the outlet flow path when either of the first valve is in a first valve closed position or the second valve is in a second valve closed position.

This application claims the benefit of U.S. Provisional Patent Application Ser. No. 63/374,046, filed Aug. 31, 2022, the entire content of which incorporated herein by reference.

### TECHNICAL FIELD

The present technology is related to delivery of a therapeutic agent to tissue of a patient.

### BACKGROUND

Some medical procedures involve the delivery of a therapeutic agent to tissue of a patient. For example, some medical ablation procedures involve ablating tissues, such as nerves, near vasculature of a patient, using a therapeutic agent. As an example, a therapeutic agent can be delivered to tissue to modify the activity of nerves at or near the target tissue site as part of neuromodulation therapy. The nerves can be, for example, sympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

### SUMMARY

The present disclosure describes medical systems configured to deliver a therapeutic agent to tissue of a patient. The medical systems are configured to help reduce or even prevent pressurization of the therapeutic agent, which can reduce a volume of the therapeutic agent delivered to tissue of the patient or even prevent the therapeutic agent from being delivered to the patient e.g., before the therapy delivery element of the system is positioned as desired at a target tissue site in a patient. As discussed below, the system is configured to control the pressurization of the therapeutic agent based on an inflation pressure of a balloon that is used to position the therapeutic element (e.g., one or more injection ports) relative to a target tissue site in the patient.

The medical system includes a therapeutic element (e.g., an injection element configured to deliver a therapeutic agent) configured to deliver the therapeutic agent to a vessel wall or other tissues of the patient via a catheter that includes a balloon. The therapeutic agent can be used to provide, for example, neuromodulation therapy, such as renal denervation. The balloon is configured to be expanded within the blood vessel to, for example, assist in positioning the therapeutic element within the blood vessel, assist in occluding the blood vessel during a medical procedure, assist in maintaining the catheter within the blood vessel, assist in displacing the catheter and a wall of the blood vessel, and/or for other reasons. For example, the expanded balloon can be configured to help hold the therapeutic element in apposition with the vessel wall, approximately center the catheter within the blood vessel, and/or assists in other ways.

In examples described herein, a medical system is configured to deliver the therapeutic agent (e.g., a chemical agent) is at a relatively high pressure. The medical system may be configured such that a clinician may cause pressurization of the therapeutic agent to facilitate the delivery to a target site in a patient. In examples, the catheter system is configured to control the pressurization of the therapeutic agent within the catheter based on the inflation pressure of the balloon to, for example, help the clinician assess a position of the therapeutic element prior to the pressurization. For example, the catheter system may be configured to substantially limit and/or prevent the pressurization of the therapeutic agent in the catheter system when an inflation pressure of the balloon is below a threshold (e.g., potentially indicating the balloon may not have the therapeutic element positioned against and/or in close proximity to the vessel wall). The catheter system may be configured to enable delivery of the therapeutic agent through the lumen of the catheter and ultimately from the lumen to tissue of a patient when the inflation pressure of the balloon is greater than or equal to the threshold (e.g., potentially indicating the balloon has likely inflated to position the therapeutic element against and/or in close proximity to the vessel wall).

In examples, the medical system includes a valve manifold configured to fluidically couple an inlet flow path and an outlet flow path to pressurize the therapeutic agent. The valve manifold includes a first valve configured to transition to a first valve open position based on an inflation pressure within an interior volume of the balloon. The valve manifold includes a second valve configured to position in a second valve open position based on the positioning (e.g., by a clinician) of an actuator. The valve manifold is configured to fluidically couple the inlet flow path and the outlet flow path (allowing pressurizing of the therapeutic agent) when both the first valve is in the first valve open position (e.g., due to the inflation pressure of the balloon) and the second valve is in the second valve open position (e.g., due to a clinician positioning the actuator to open the second valve). The valve manifold is configured to fluidically isolate the inlet flow path and the outlet flow path (substantially preventing pressurizing of the therapeutic agent) when either of the first valve is in a first valve closed position (e.g., due to relatively low inflation pressure of the balloon) or the second valve is in a second valve closed position (e.g., due to the clinician leaving the second valve in the closed position, and/or positioning the actuator to close the second valve).

In examples, a medical system comprises: a first valve biased to a first valve closed position; a valve manifold defining an inlet flow path and an outlet flow path, the valve manifold defining a pressure chamber configured to cause a pressure of a fluid in the pressure chamber to cause the first valve to move from the first valve closed position to a first valve open position; a second valve; and an actuator configured to cause the second valve to position in one of a second valve open position or a second valve closed position, wherein the first valve and the second valve are configured to fluidically couple the inlet flow path and the outlet flow path when the pressure of the fluid causes the first valve to move to the first valve open position and the actuator causes the second valve to position in the second valve open position, and wherein the first valve and the second valve are configured to fluidically isolate the inlet flow path and the outlet flow path when the first valve is in the first valve closed position or the actuator causes the second valve to position in the second valve closed position.

In examples, a method comprises: pressurizing, using an inflation catheter, an interior volume of a balloon using an inflation fluid, wherein the inflation fluid is configured to establish a pressure in a pressure chamber of a valve manifold, the pressure being sufficient to cause a first valve to move from a first valve closed position to a first valve open position, the first valve being biased to the first valve closed position; and fluidically coupling an inlet flow path of a valve manifold to an outlet flow path of the valve manifold by at least transitioning, using an actuator, a second valve from a second valve closed position to a second valve open position, wherein the first valve and the second valve are configured to fluidically couple the inlet flow path and the outlet flow path when the first valve is in the first valve open position and the second valve is in the second valve open position, and wherein the first valve and the second valve are configured to fluidically isolate the inlet flow path and the outlet flow path valve when at least one the first valve is in the first valve closed position or second valve is in the second valve closed position.

Further disclosed herein is a medical system that is configured to deliver a therapeutic agent to a patient using a catheter system having a balloon, wherein a valve manifold includes a first valve configured to transition to a first valve open position based on an inflation pressure of the balloon, wherein the valve manifold includes a second valve configured to position in a second valve open using an actuator, wherein the valve manifold is configured to fluidically couple an inlet flow path and the outlet flow path to pressurize of the therapeutic agent when the first valve is in the first valve open position and the second valve is in the second valve open position, wherein the valve manifold is configured to fluidically isolate the inlet flow path and the outlet flow path when either of the first valve is in a first valve closed position or the second valve is in a second valve closed position.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic illustration of an example medical system including a medical device configured to deliver a therapeutic agent to tissue of a patient.

FIG. 2 is a schematic illustration of the medical system of FIG. 1 within a blood vessel of a patient.

FIG. 3 is a schematic illustration of the medical system of FIG. 1 accessing a renal artery of a patient.

FIG. 4 is a schematic illustration of the medical system of FIG. 1 in a delivery configuration.

FIG. 5 is a schematic illustration of a balloon of the medical system of FIG. 1 in an expanded configuration, with a first valve in a first valve open position and a second valve in a second valve closed position.

FIG. 6 is an example illustration depicting a pressure within and a dimension defined by the balloon of the medical system of FIG. 1, with the first valve in the first valve open position and the second valve in a second valve open position.

FIG. 7 is a perspective view of an example handle of the medical system of FIG. 1.

FIG. 8 is a perspective cross-sectional view of an example valve manifold of the medical system of claim 1.

FIG. 9 is a flow diagram illustrating an example technique for pressurizing an interior volume of a balloon using the medical system of FIG. 1.

### DETAILED DESCRIPTION

The present technology is directed to devices, systems, and methods for delivering a therapeutic agent to tissue of a patient. The therapeutic agent can be, for example, a therapeutic agent used for neuromodulation, such as renal neuromodulation.

Conditions such as arrhythmias, hypertension, states of volume overload (e.g., heart failure), and progressive renal disease due to excessive activation of the renal sympathetic nervous system (SNS), may be mitigated by modulating the activity of overactive nerves (neuromodulating), for example, denervating or reducing the activity of the overactive nerves. Sympathetic nerves of the kidneys terminate in the renal blood vessels, the juxtaglomerular apparatus, and the renal tubules, among other structures. The overactive nerves may be denervated by ablating sympathetic nerve tissue in or near renal blood vessels, e.g., using a therapeutic agent alone in combination with other ablation modalities. The therapeutic agent may be delivered to the sympathetic tissue via navigating a catheter including one or more therapeutic element (e.g., injection ports) within the vasculature of the patient. In the case of chemical denervation (e.g., chemical renal denervation), the medical system may be configured to pressurize the therapeutic agent to facilitate delivery of the therapeutic agent to a target treatment site. The therapeutic agent can be pressurized to, for example, enable the therapeutic agent to penetrate through the vessel wall to reach nerves proximate the vessel wall.

Renal neuromodulation, such as renal denervation, may be accomplished using one or more of a variety of treatment modalities, including radio frequency (RF) energy, microwave energy, ultrasound energy, a therapeutic agent, or the like. When using a therapeutic agent, a neuromodulation catheter may be delivered to a renal vessel, such as a renal artery, of a patient. The neuromodulation catheter may include at least one port or needle through which the therapeutic agent is delivered. The therapeutic agent may be selected to modulate activity of one or more renal nerves adjacent to the renal artery in which the neuromodulation catheter is positioned. For example, the therapeutic agent may be a neurotoxic chemical selected to chemically ablate the one or more renal nerves near the renal artery. The therapeutic agent may include one or more chemical agents, such as, but not limited to, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof.

The present disclosure describes example medical systems that include at least one balloon configured to be positioned within a patient (e.g., within a blood vessel of the patient) and at least one therapeutic element configured to deliver therapy to a target tissue site of the patient to achieve a therapeutic outcome. The medical systems, as well as systems including the medical systems and methods of using the medical system, can be used for any suitable medical procedures that include delivering a therapeutic agent alone in combination with other therapy (e.g., ultrasound energy, microwave energy, cryotherapy, and/or radiofrequency (RF) energy) to a target tissue site via the therapeutic element. Thus, although neuromodulation (e.g., denervation) is primarily referred to herein, the devices, systems, and methods described herein can be used with other medical procedures. The medical system is configured to be positioned (e.g., by a clinician) within a blood vessel or other anatomical lumen of a patient. The at least one balloon is configured to be expanded within the anatomical lumen to, for example, assist in positioning the therapeutic element within the anatomical lumen, assist in occluding the anatomical lumen, assist in maintaining an elongate body of the catheter within the anatomical lumen, assist in displacing the elongate body and a wall of the anatomical lumen, and/or for other reasons.

The balloon is configured to inflate within the anatomical lumen to position the therapeutic element against and/or in close proximity to a wall of the anatomical lumen. As discussed in further detail below, the medical system is configured to help reduce or even prevent pressurization of the therapeutic agent based on an inflation state of the balloon. This can reduce a volume of the therapeutic agent delivered to tissue of the patient or even prevent the therapeutic agent from being delivered to the patient before the therapy delivery element of the system is positioned as desired at a target tissue site in a patient.

In examples, the therapeutic element includes an injection component configured to deliver a therapeutic agent, such as a chemical ablative agent and/or another therapeutic agent, to a target treatment site in a patient. The injection component may be, for example, a needle, a needleless injection tube defining one or more injection ports, and/or another component configured to deliver the therapeutic agent. In examples, the balloon is configured to inflate within the anatomical lumen to position the injection component against and/or in close proximity to a vessel wall as the balloon inflates. Hence, the balloon may assist in substantially establishing and/or maintaining the therapeutic element in a position relative to the target site in preparation for and/or during delivery of the therapeutic agent to the target site.

In examples described herein, the catheter system is configured to pressurize the therapeutic agent at a location proximal to the injection component to facilitate and cause delivery of the therapeutic agent through a lumen of the catheter to the target site. The catheter system is configured to substantially limit and/or prevent the pressurization based on the inflation pressure of the balloon to, for example, help the clinician assess a position of the therapeutic element prior to the pressurization. For example, the catheter system may be configured to substantially limit and/or prevent pressurization of the therapeutic agent when an inflation pressure of the balloon is below a threshold (e.g., potentially indicating the balloon may not have the therapeutic element positioned against and/or in close proximity to the vessel wall). The catheter system may be configured to permit pressurization of the therapeutic agent when the inflation pressure of the balloon is greater than or equal to the threshold (e.g., potentially indicating the balloon has likely inflated to position the therapeutic element against and/or in close proximity to the vessel wall).

In examples, the catheter system is configured to pressurize the therapeutic agent using a pressurization fluid such as pressurized carbon dioxide (CO₂). For example, the medical system may be configured such that the pressurization fluid acts on a pressurizing element (e.g., a piston or diaphragm) to cause the pressurizing element to pressurize the therapeutic agent. In some examples, the catheter system includes a valve manifold configured to receive the pressurization fluid via an inlet flow path and discharge the pressurization fluid (e.g., to the pressurization element) via an outlet flow path. The valve manifold is configured to fluidically couple the inlet flow path and the outlet flow path (e.g., such that the pressurization fluid may act on the pressurizing element) when both the inflation pressure of the balloon indicates a sufficient pressure and a clinician acts (e.g., by positioning an actuator) to cause the fluidic coupling. The valve manifold is configured to substantially prevent the fluidic coupling when either the inflation pressure of the balloon is insufficient (e.g., below a threshold), or the clinician has not taken the necessary action to cause the fluidic coupling (e.g., has not positioned the actuator). Thus, the valve manifold is configured to coordinate the inflation pressure of the balloon and the action of the clinician to, for example, assist the clinician in assessing a position of the therapeutic element prior to pressurizing the therapeutic agent. For example, the medical system may assist the clinician in assessing when the balloon has likely inflated sufficiently to position the therapeutic element against and/or in close proximity to the vessel wall, such that positioning the actuator (e.g., by the clinician) will cause pressurization of the therapeutic agent.

In some examples, the valve manifold includes a first valve and a second valve configured to fluidically couple the inlet flow path and the outlet flow path, such that the pressurization fluid may flow through the valve manifold. The valve manifold may be configured such that fluidically coupling the inlet flow path and the outlet flow path (e.g., such that the pressurization fluid acts on the pressurizing element) requires both the first valve and the second valve to be in an open position (e.g., a fully open or a partially open state). In some examples, the valve manifold is configured such that if either of the first valve or the second valve is in a closed position, the valve manifold fluidically isolates the inlet flow path and the outlet flow path, such that the pressurization fluid is substantially prevented (e.g., prevented or nearly prevented to the extent permitted by manufacturing tolerances) from flowing through the valve manifold (e.g., substantially prevented from acting on the pressurizing element) and pressurizing the therapeutic agent. The prevention of the pressurization of the therapeutic agent may reduce a volume of therapeutic agent unintentionally delivered to a lumen of the catheter and, ultimately to the patient, or even prevent the therapeutic agent from being delivered to the lumen of the catheter.

For example, the first valve may be configured to fluidically isolate the inlet flow path and the outlet flow path when the first valve is in a closed position, regardless of the position of the second valve. The second valve may be configured to fluidically isolate the inlet flow path and the outlet flow path when the second valve is in a closed position, regardless of the position of the first valve. Hence, the valve manifold is configured to fluidically isolate the inlet flow path and the outlet flow path if either of the first valve or the second valve is in a closed position. In some examples, the valve manifold is configured such that the first valve and the second valve are fluidically coupled in series when the manifold fluidically couples the inlet flow path and the outlet flow path.

In examples, the valve manifold is configured such that an inflation pressure within an interior volume of the balloon greater than or equal to a predetermined balloon inflation pressure is sufficient to overcome a biasing of the first valve. For example, the first valve may be biased in a first valve closed position, such that the inlet flow path and the outlet flow path are fluidically isolated. The first valve is configured to transition from the first valve closed position to a first valve open position based on the inflation pressure of the balloon. In examples, the first valve is configured to transition from the first valve closed position to the first valve open position based on a pressure of a control fluid (e.g., a fluid separate from the pressurization fluid used to pressurize the therapeutic agent). The control fluid can be, for example, the inflation fluid used to inflate the balloon, such that the pressure chamber can be in fluid communication with an interior volume of the balloon and configured to receive the inflation fluid from the balloon.

In some examples, the valve manifold includes a pressure chamber configured such that a pressure of the control fluid is indicative of (e.g., based on or a function of) the inflation pressure of the balloon. The first valve may be configured to transition from the first valve closed position to the first valve open position when the pressure of the control fluid acting on the first valve and/or another component (e.g., a diaphragm) overcomes the biasing of the first valve in the first valve closed position. In examples, the valve manifold includes a biasing element such as a spring or other compressive element configured to bias the first valve in the first valve closed position. Hence, the valve manifold is configured to transition the first valve from the first valve closed position to the first valve open position when the inflation pressure of the balloon sufficiently increases to overcome the biasing of the first valve, such that the position of the first valve is dependent on the inflation pressure of the balloon.

The second valve is configured to transition between a second valve closed position and a second valve open position based on an action of a clinician. For example, the second valve may include an actuator (e.g., a manual actuator that is manipulated by the clinician) configured to transition the second valve between a second valve closed position and a second valve open position. The valve manifold is configured to fluidically isolate the inlet flow path and the outlet flow path when the second valve is in the second valve closed position regardless of the position of the first valve, such that the clinician may control the flow of the pressurization fluid through the manifold. In examples, the actuator is configured to transition the second valve between the second valve closed position and the second valve open position based on a physical manipulation of a component (e.g., by the clinician), such as physical manipulation of a mechanical element configured to physically translate the second valve, physical manipulation of a button or switch configured to communication a valve position signal to the second valve, and/or another physical manipulation. Hence, the valve manifold may be configured such that fluidically coupling the inlet flow path and the outlet flow path to pressurize the therapeutic agent substantially requires a pressure in the balloon sufficient to place the first valve in the first valve open position, as well as an action by the clinician to cause the actuator to place the second valve in the second valve open position.

The valve manifold is configured to fluidically isolate the inlet flow path and the outlet flow path when at least one of the first valve or the second valve transitions from an open position to a closed position. For example, the first valve (e.g., due to the biasing of the first valve in the first valve closed position) may be configured to transition from the first valve open position to the first valve closed position when an inflation pressure in the balloon decreases below a threshold pressure value. The valve manifold may fluidically isolate the inlet flow path and the outlet flow path when the first valve positions in the first valve closed position, such that the pressurization fluid is substantially unable to pressurize and/or continue pressurizing the therapeutic agent. Hence, the valve manifold may be configured to prevent and/or substantially reduce pressurization of the therapeutic agent when the inflation pressure of the balloon decreases below the threshold pressure value (e.g., due to a partial and/or substantially complete deflation of the balloon when the balloon is positioned within an anatomical lumen).

Similarly, the valve manifold may be configured to prevent and/or substantially secure pressurization of the therapeutic agent when the second valve is positioned in the second valve closed position. For example, the valve manifold may be configured such that an action by a clinician (e.g., a manipulation of the actuator) causes the valve manifold to fluidically isolate the inlet flow path and the outlet flow path, such that the clinician may prevent and/or substantially secure pressurization of the therapeutic agent regardless of the inflation pressure of the balloon.

In examples, the medical system includes a switch configured to substantially prevent the actuator from causing the second valve to transition from the second valve closed position to the second valve open position. The medical system may be configured such causing the second valve to transition from the second valve open position to the second valve closed position requires altering (e.g., by a clinician) a position of the switch. For example, the switch may be configured to include a locked position and a firing position. The switch may be configured to substantially prevent the actuator from causing the second valve to transition to the second valve open position when in the locked position. The switch may be configured to allow the actuator to cause the second valve to transition to the second valve open position when in the firing position. In some examples, the switch is biased (e.g., spring loaded) in the locked position, such that an operation of the actuator to cause the second valve to position in the second valve open position first requires a manipulation of the switch (e.g., by a clinician) from the locked position to the firing position. Hence, the medical system may be configured such that a clinician is required to initially manipulate the biased switch before causing the valve manifold to fluidically couple the inlet flow path and the outlet flow path. The required sequential operation of the switch and the actuator may provide assurance to the clinician that pressurization of the therapeutic agent may only occur based on deliberate actions of the clinician.

Hence, in examples described herein, a medical system includes a balloon and a catheter configured to deliver a pressurized therapeutic agent to a target site in an anatomical lumen of a patient. The medical system is configured such that a clinician may cause pressurization of the therapeutic agent using a pressurization fluid such as pressurized carbon dioxide (CO₂)). The medical system includes a valve manifold configured to receive the pressurization fluid via an inlet flow path and discharge the pressurization fluid to the pressurization element via an outlet flow path. The valve manifold is configured to fluidically couple the inlet flow path and the outlet flow path when both the inflation pressure of the balloon indicates a sufficient pressure (e.g., greater than or equal to a predetermined threshold pressure value), and the clinician acts (e.g., by positioning a manual actuator) to cause the fluidic coupling. Thus, the valve manifold may be configured to substantially coordinate the inflation pressure of the balloon and the action of the clinician to, for example, assist the clinician in assessing a position of the therapeutic element prior to pressurizing the therapeutic agent.

While blood vessels are primarily referred to throughout the disclosure, the devices, systems, and techniques described herein are also applicable to other target tissue sites. In addition, while a single balloon is primarily referred to throughout the disclosure, medical systems described herein can include more than one balloon, such as two, three, four or more balloons. In some examples, the balloon is configured to expand to a range of dimensions (e.g., diameters). For example, the balloon can be a compliant balloon. The balloon may be configured to expand to define a particular dimension within the range based on an inflation pressure within the balloon. In some examples, control circuitry of the medical system is configured to control a dimension (also referred to herein as a size, e.g., a diameter) of the balloon by at least controlling the inflation pressure of the balloon. In this way, the size of the balloon may be adjusted to account for the size of the blood vessel in which the balloon is positioned (e.g., adjusted such that a diameter of the inflated balloon enables to balloon to be in apposition with a blood vessel wall during a procedure). As used herein, a pressure of a fluid (e.g., an inflation pressure) may refer to a static pressure of the fluid, a dynamic pressure of the fluid, or a total pressure of the fluid.

Although the present technology is herein described in many instances with reference to renal nerves and vessels, the present technology also has application to neuromodulation at other anatomical sites (e.g., spinal neuromodulation, cardiac neuromodulation, brain neuromodulation, sacral neuromodulation, urinary neuromodulation, and/or neuromodulation techniques directed to other portions of a body) and their associated nerves and that such devices and systems can be configured (e.g., have suitable shape and dimensions) for such sites. For example, a catheter may be configured to deliver energy with a portion of the catheter carrying a therapeutic element positioned with a particular anatomical lumen or a particular tissue (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures).

As used herein, the terms “distal” and proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

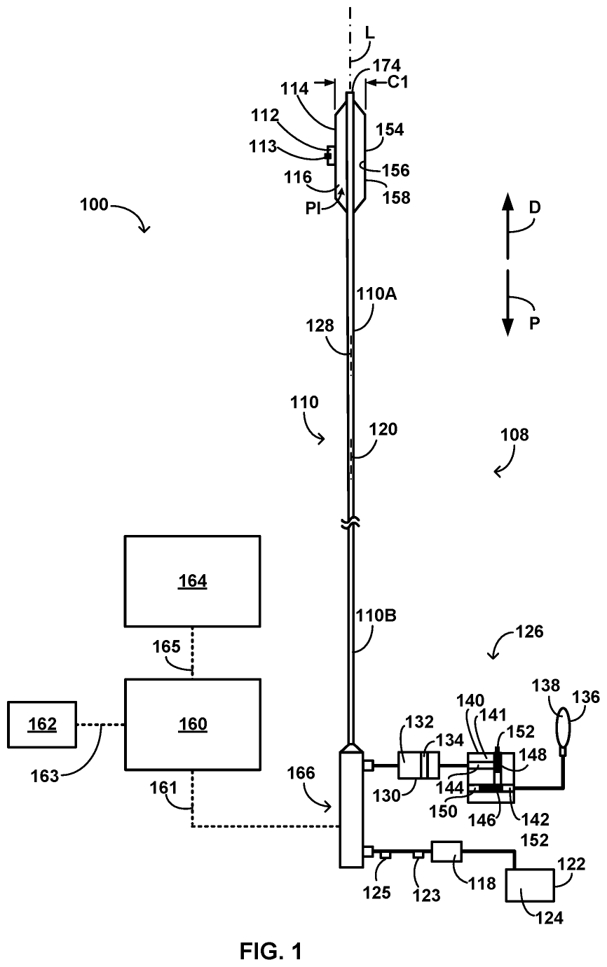

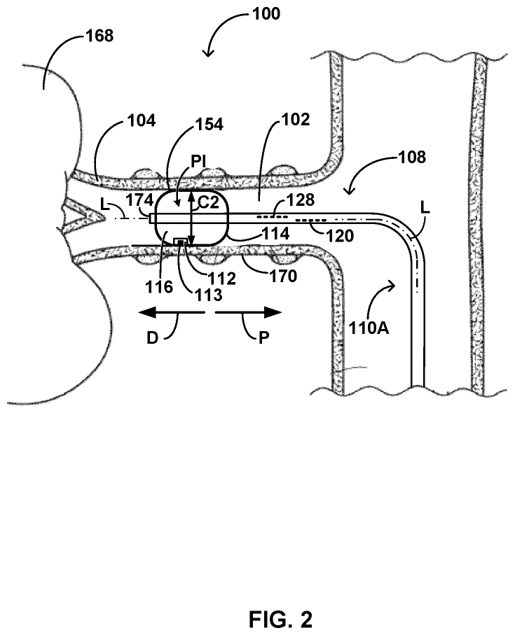

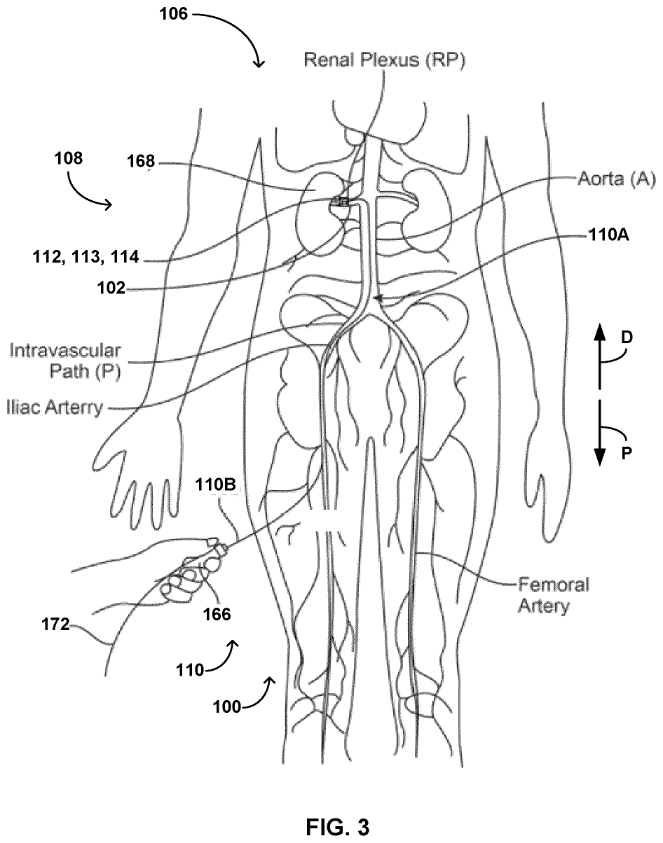

FIG. 1 is a partially schematic view of an example medical system 100 configured in accordance with examples of the present disclosure. FIG. 2 is a schematic illustration of a portion of medical system 100 within a blood vessel 102 of a patient 106, the blood vessel 102 having a vessel wall 104. FIG. 3 illustrates medical system 100 being navigated through vasculature of a patient 106 to a target treatment site within blood vessel 102. In the examples of FIGS. 2 and 3, blood vessel 102 is a renal artery and vessel wall 104 is a renal artery wall. However, in other examples, medical system 100 is be configured to deliver treatments to other blood vessels, anatomical lumens, and/or other tissues, such as an external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures of a patient.

In examples, medical system 100 is configured to provide neuromodulation to patient 106. Neuromodulation (e.g., renal neuromodulation) is the partial or complete incapacitation or other effective disruption of nerves (e.g., nerves terminating in the kidneys or in structures closely associated with the kidneys). Neuromodulation can include inhibiting, reducing, and/or blocking neural communication along neural fibers (e.g., efferent and/or afferent neural fibers). Such incapacitation can be long-term (e.g., permanent or for periods of months, years, or decades) or short-term (e.g., for periods of minutes, hours, days, or weeks). Neuromodulation may be expected to contribute to the systemic reduction of sympathetic tone or drive and/or to benefit at least some specific organs and/or other bodily structures innervated by sympathetic nerves. Accordingly, neuromodulation may be expected to be useful in treating clinical conditions associated with systemic sympathetic overactivity or hyperactivity, particularly conditions associated with central sympathetic overstimulation.

For example, renal neuromodulation may be expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions. The renal neuromodulation can be electrically induced, thermally-induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen (e.g., proximate to vessel wall 104 of blood vessel 102). For example, with regard to a renal artery, a treatment procedure can include modulating nerves in the renal plexus, which lay intimately within or adjacent to the adventitia of the renal artery.

For ease of description, the following discussion will be primarily focused on delivering a therapeutic agent, such as a neurotoxic chemical, to the target site. However, medical system 100 (e.g., therapeutic element 112) may include elements or structures configured to deliver other types of therapy. For example, medical system 100 may include one or more electrodes, one or more ultrasound transducers, a cryoablation element (e.g., a balloon), and/or other components configured to deliver energy for ablation of nerves.

Medical system 100 includes a catheter system 108. Catheter system 108 includes a therapeutic element 112 configured to deliver a therapy to tissue to, for example, conduct a neuromodulation or another procedure on vessel wall 104 and/or other tissues associated with blood vessel 102. Once at a target site in patient 106, therapeutic element 112 can be configured to deliver therapy using, for example, a therapeutic agent alone or in combination with RF energy, microwave energy, ultrasound energy, or the like to provide or facilitate neuromodulation therapy at the target site. For example, therapeutic element 112 may include an injection element 113 configured to deliver a therapeutic agent to ablate tissue at a target treatment site. Injection element 113 may be, for example, a needle, an injection tube defining a needleless injection port, and/or another component configured to deliver the therapeutic agent to the target site.

In some examples, therapeutic element 112 is configured to at least partially pierce and/or puncture, or cause a therapeutic agent to at least partially pierce and/or puncture, vessel wall 104 of blood vessel 102 to cause delivery the therapeutic agent to a target site. In other examples, therapeutic element 112 does not include a needle and is configured to deliver a relatively high pressure therapeutic agent to vessel wall 104 to cause delivery the therapeutic agent through at least some tissue of vessel wall 104 to a target site. For example, therapeutic element 112 may be configured to deliver the therapeutic agent to the adventitia and/or peri-adventitia, in which renal nerves are located. In examples, the therapeutic agent is selected to neuromodulate (e.g., chemically ablate) nerve tissue of the renal plexus adjacent to a renal artery. The therapeutic agent may include, for example, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof. In some examples, the therapeutic agent may be heated or cooled to additionally or alternatively thermally ablate nerve tissue (e.g., nerve tissue of the renal plexus adjacent to renal artery). For some needleless injection examples, system 100 is configured to pressurize the therapeutic agent to 5 Megapasal (Mpa) to 18 Mpa (e.g., 750-2500 pounds per square inch (psi)), such as about 10 Mpa (e.g., about 1500 psi), for delivery to the tissue via therapeutic element 112 (e.g., one or more injection ports).

Catheter system 108 includes an catheter body 110 configured to be positioned (e.g., by a clinician) within blood vessel 102 of patient 106. Catheter system 108 includes one or more expandable elements such as balloon 114 (“balloon 114”) configured to expand (e.g., by inflation) when catheter body 110 is positioned within blood vessel 102. Balloon 114 may be configured to expand to, for example, assist in positioning therapeutic element 112 against and/or in close proximity to vessel wall 104, assist in occluding blood vessel 102 during a procedure, assist in maintaining catheter body 110 within blood vessel 102, assist in displacing and/or maintaining a displacement between catheter body 110 and vessel wall 104, and/or for other reasons. Catheter body 110 defines a longitudinal axis L. Balloon 114 may be configured to expand radially outwards relative to longitudinal axis L (e.g., substantially perpendicular to longitudinal axis L) when balloon 114 is inflated within blood vessel 102 of patient 106. In examples, catheter body 110 supports (e.g., mechanically supports) balloon 114. In examples, balloon 114 supports (e.g., mechanically supports) therapeutic element 112. Therapeutic element 112 may be positioned substantially external to balloon 114, such as on an outer surface of balloon 114, and/or positioned substantially internal to balloon 114, such as in an interior volume 116 of balloon 114 (balloon interior volume 116″).

Catheter system 108 is configured to control an inflation pressure PI within balloon interior volume 116 to control a size (e.g., a diameter) of balloon 114. For example, catheter system 108 may be configured to inject an inflation fluid (e.g., saline) into balloon interior volume 116 to control the inflation pressure. In examples, catheter system 108 includes an inflation fluid delivery system 118 such as pump or syringe configured to drive the inflation fluid to balloon interior volume 116. Catheter system 108 (e.g., catheter body 110) may define an inflation fluid flow path 120 (e.g., an inflation lumen) configured to fluidically couple inflation fluid delivery system 118 and balloon interior volume 116. In examples, medical system 100 includes a inflation fluid container 122 (e.g., a syringe) defining a reservoir 124 configured to hold a volume of the inflation fluid. Inflation fluid delivery system 118 may be configured to draw the fluid from reservoir 124 and discharge the fluid into inflation fluid flow path 120. In examples, catheter system 108 may be configured to commence, cease, adjust and/or substantially establish a flow rate of the inflation fluid in order to adjust and/or establish the inflation pressure PI within balloon interior volume 116, and thus adjust and/or establish a size of balloon 114.

In some examples, catheter system 108 includes a flow sensor 123 configured to sense a parameter indicative of a flow rate (e. indicative of a mass flow rate) provided by inflation fluid delivery system 118 to inflation fluid flow path 120. In some examples, catheter system 108 includes a sensor 125 configured to sense a parameter indicative of the inflation pressure PI of balloon 114.

Catheter system 108 is configured to deliver a therapeutic agent to therapeutic element 112 such that, for example, therapeutic element 112 (e.g., injection element 113) delivers the therapeutic agent to a target site. In examples, catheter system 108 includes a therapeutic agent delivery system 126 configured to drive the therapeutic agent to therapeutic element 112 (e.g., injection element 113). Catheter system 108 (e.g., catheter body 110) defines a therapeutic agent flow path 128 (e.g., a catheter lumen) configured to fluidically couple therapeutic agent delivery system 126 and therapeutic element 112. In examples, therapeutic agent delivery system 126 includes a therapeutic agent container 130 defining a therapeutic agent reservoir 132 configured to hold a volume of the therapeutic agent. Therapeutic agent delivery system 126 may be configured to fluidically couple therapeutic agent reservoir 132 and therapeutic agent flow path 128. In examples, therapeutic agent delivery system 126 is configured to substantially discharge the therapeutic agent from therapeutic agent reservoir 132 to therapeutic agent flow path 128.

Therapeutic agent delivery system 126 is configured to pressurize the therapeutic agent to discharge the therapeutic agent from therapeutic agent reservoir 132 into therapeutic agent flow path 128. In examples, therapeutic agent delivery system 126 includes a pressurization element 134 configured to pressurize (e.g., substantially increase a pressure of) a therapeutic agent within therapeutic agent reservoir 132. Therapeutic agent delivery system 126 may be configured such that the pressurization causes the therapeutic agent within therapeutic agent reservoir 132 to discharge from therapeutic agent reservoir 132 into therapeutic agent flow path 128. Therapeutic agent flow path 128 is configured to receive the pressurized therapeutic agent from therapeutic agent delivery system 126 and provide the pressurized therapeutic agent to therapeutic element 112 (e.g., injection element 113).

Therapeutic agent delivery system 126 is be configured such that a clinician may cause pressurization of the therapeutic agent to cause delivery to the target site. In examples, therapeutic agent delivery system 126 is configured to pressurize the therapeutic agent using a pressurization fluid such as pressurized carbon dioxide (CO₂)). In examples, therapeutic agent delivery system 126 includes a pressurization fluid container 136 defining a pressurization fluid reservoir 138 configured to hold a volume of the pressurization fluid. Therapeutic agent delivery system 126 may be configured such that, when the pressurization fluid acts on pressurization element 134 (e.g., exerts a fluid pressure on pressurization element 134), pressurization element 134 pressurizes the therapeutic agent within therapeutic agent reservoir 132.

Catheter system 108 (e.g., therapeutic agent delivery system 126) is configured to coordinate the inflation pressure of balloon 114 and the action of the clinician to, for example, assist the clinician in assessing a position of the therapeutic element prior to pressurizing the therapeutic agent (which ultimately causes delivery of the therapeutic agent to therapeutic agent flow path 128 and out via therapeutic element 112. In the example shown in FIG. 1, therapeutic agent delivery system 126 includes a valve manifold 140 configured to receive the pressurization fluid (e.g., from pressurization fluid container 136) via an inlet flow path 142. Valve manifold 140 is configured to discharge the pressurization fluid (e.g., to pressurization element 134) via an outlet flow path 144 when valve manifold 140 fluidically couples inlet flow path 142 and outlet flow path 144.

In examples, valve manifold 140 includes a manifold body 141 defining inlet flow path 142 and/or outlet flow path 144. Therapeutic agent delivery system 126 is configured to fluidically couple inlet flow path 142 and outlet flow path 144 when an inflation pressure of balloon 114 indicates a sufficient pressure and a clinician acts (e.g., by positioning an actuator) to cause the fluidic coupling. Therapeutic agent delivery system 126 is configured to substantially prevent the fluidic coupling of inlet flow path 142 and outlet flow path 144 when either the inflation pressure of the balloon may be insufficient (e.g., below a threshold), or the clinician has not taken the necessary action to cause the fluidic coupling (e.g., has not positioned the actuator).

In examples, therapeutic agent delivery system 126 includes a first valve 146 and a second valve 148 configured to fluidically couple inlet flow path 142 and outlet flow path 144. First valve 146 and second valve 148 are configured to fluidically couple and/or fluidically isolate inlet flow path 142 and outlet flow path 144 based on the respective positions of first valve 146 and second valve 148. In examples, first valve 146 is configured to establish an open position (“first valve open position”) and a closed position (“first valve closed position”). First valve 146 is configured to allow a flow through first valve 146 in the first valve open position and substantially block (e.g., fully block or fully block to the extent permitted by manufacturing tolerances) the flow through first valve 146 in the first valve closed position. Similarly, second valve 148 is configured to allow a flow through second valve 148 in the second valve open position and substantially block the flow through second valve 148 in the second valve closed position.

Therapeutic agent delivery system 126 is configured such that, when first valve 146 is in the first valve open position and second valve 148 is in the second valve open position, valve manifold 140 fluidically couples inlet flow path 142 and outlet flow path 144 (e.g., such that the pressurization fluid may act on pressurization element 134 to pressurize the therapeutic agent). Therapeutic agent delivery system 126 is configured such that, if either of first valve 146 is in the first valve closed position or second valve 148 is in the second valve closed position, valve manifold 140 fluidically isolates inlet flow path 142 and outlet flow path 144 (e.g., such that the pressurization fluid is substantially prevented (e.g., prevented or nearly prevented to the extent permitted by manufacturing tolerances) from flowing through valve manifold 140 to act on pressurization element 134).

Therapeutic agent delivery system 126 is configured such that a position of first valve 146 is dependent on an inflation pressure within balloon interior volume 116. In examples, first valve 146 is configured to transition from the first valve closed position to the first valve open position (and vice-versa) based on a pressure of a control fluid within a pressure chamber 150 defined by valve manifold 140. Valve manifold 140 may be configured such that the pressure of the control fluid is dependent on (e.g., directly proportional to) the inflation pressure within balloon interior volume 116 of balloon 114. For example, therapeutic agent delivery system 126 may be configured to fluidically and/or mechanically couple balloon interior volume 116 and pressure chamber 150, such that a pressure of the inflation fluid within balloon interior volume 116 causes a pressure to be exerted on the control fluid within pressure chamber 150. In examples, the control fluid within pressure chamber 150 comprises at least a portion of the inflation fluid. Thus, therapeutic agent delivery system 126 is configured to transition first valve 146 from the first valve closed position to the first valve open position (such that first valve 146 may allow a flow of pressurization fluid from pressurization fluid reservoir 138 substantially through first valve 146) based on the inflation pressure within balloon interior volume 116.

In some examples, therapeutic agent delivery system 126 is configured such that a position of second valve 148 is dependent on (e.g., controlled by) an action of a clinician. In examples, second valve 148 includes an actuator 152 configured to transition second valve 148 between the second valve closed position and the second valve open position. Therapeutic agent delivery system 126 is configured to fluidically isolate inlet flow path 142 and outlet flow path 144 when second valve 148 is in the second valve closed position regardless of the position of first valve 146, such that the clinician may control the flow of pressurization fluid from pressurization fluid reservoir 138 to pressurization element 134. For example, actuator 152 may be configured to transition second valve 148 between the second valve closed position and the second valve open position based on a physical manipulation (e.g., by the clinician) of a mechanical element (e.g., a manual valve operator), manipulation of a button or switch, and/or another physical manipulation. Hence, therapeutic agent delivery system 126 is configured such that fluidically coupling inlet flow path 142 and outlet flow path 144 to cause pressurization of the therapeutic agent substantially requires a pressure in balloon 114 sufficient to place first valve 146 in the first valve open position, as well as an action by the clinician to cause actuator 152 to place second valve 148 in the second valve open position.

Catheter body 110 defines a distal portion 110A (“distal body portion 110A”) and a proximal portion 110B (“proximal body portion 110B”). Therapeutic element 112 and/or balloon 114 are positioned on distal body portion 110A in the example shown in FIG. 1. In examples, catheter system 108 is configured to assume a relatively low profile delivery configuration in which at least one of distal body portion 110A and/or balloon 114 defines a maximum cross-sectional dimension C1 (e.g., a diameter), which can be measured in a direction perpendicular to longitudinal axis L. The dimension C1 may define a displacement sufficient to allow the passage of at least distal body portion 110A and balloon 114 through vasculature of patient 106 to reach a target treatment site within patient 106. In some examples, distal body portion 110A is configured to locate balloon 114 and/or therapeutic element 112 at an intraluminal (e.g., intravascular) location. In examples, catheter system 108 is configured such that, in the delivery configuration, the dimension C1 measures 2, 3, 4, 5, 6, or 7 French or another suitable size. Balloon 114 is configured to expand from the delivery configuration to an expanded configuration (FIG. 2) to, for example, position and/or stabilize distal body portion 110A and/or therapeutic element 112 when distal body portion 110A locates therapeutic element 112 at the target treatment site.

In some examples, balloon 114 supports therapeutic element 112, such that, for example, inflation of balloon 114 decreases a displacement between therapeutic element 112 and vessel wall 104. For example, therapeutic element 112 may be supported by an exterior surface 154 of balloon 114 (“balloon exterior surface 154”), e.g., an injection tube on an outer surface of balloon 14, and/or some portion of a balloon body 158 between and/or defining balloon exterior surface 154 and/or an interior surface 156 of balloon 114 (“balloon interior surface 156”). In some examples, catheter body 110 may be configured to support therapeutic element 112 at a fixed longitudinal location (measured along longitudinal axis L) on catheter body 110 relative to balloon 114. In some examples, catheter body 110 supports therapeutic element 112 such that therapeutic element 112 is positioned distal to balloon 114 (e.g., displaced from balloon 114 in the direction D) or proximal to balloon 114 (e.g., displaced from balloon 114 in the direction P).

In examples, medical system 100 includes control circuitry 160 configured to control an inflation pressure of the inflation fluid within balloon interior volume 116. In some examples, control circuitry 160 is configured to receive a pressure signal indicative of the inflation pressure (e.g., from sensor 125) and cause an adjustment in the inflation pressure based on the pressure signal. For example, control circuitry 160 can be configured to receive an input indicative of a pressure setpoint, and cause an adjustment in the inflation pressure (e.g., using inflation fluid delivery system 118) based on a pressure indicated by the pressure signal and the pressure setpoint. Control circuitry 160 may be configured to communicate with catheter system 108 (e.g., via communication link 161) to receive the pressure signal, adjust the inflation pressure, and/or control and/or direct other functions of catheter system 108. In other examples, an inflation pressure of the inflation fluid within balloon interior volume 116 is manually controlled by a clinician, rather than automatically by control circuitry 160.

In some examples, catheter system 108 includes a user interface 162 configured to receive the input indicative of the pressure setpoint from a user and provide the input to control circuitry 160 (e.g., via communication link 163). User interface 162 may be configured to receive the indicative input from a clinician and/or other user, such that the clinician and/or other user may specify a desired size for balloon 114. User interface 162 can have any suitable configuration sufficient to receive an input from a user. For example, user interface 162 can include a button or keypad, a touch screen, a speaker configured to receive voice commands from a user, a positioning member (e.g., a dial and/or other positioning member), and/or a display, such as a liquid crystal (LCD), light-emitting diode (LED), or organic light-emitting diode (OLED). In some examples, user interface 162 is configured to display information, such as one or more desired sizes and/or setpoints (e.g., the current desired size and/or setpoint being used by control circuitry 160 or one or more predetermined desired sizes and/or setpoints from which the user can select to input a desired size of balloon 114).

Control circuitry 160, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, control circuitry 160 includes multiple components, such as any combination of one or more microprocessors, one or more DSPs, one or more ASICs, or one or more FPGAs, as well as other discrete or integrated logic circuitry, and/or analog circuitry.

Although not shown in FIG. 1, system 100 can also include memory configured to store program instructions, such as software, which may include one or more program modules, which are executable by control circuitry 160. When executed by control circuitry 160, such program instructions may cause control circuitry 160 to provide the functionality ascribed to control circuitry 160 herein. The program instructions may be embodied in software and/or firmware. The memory can include any volatile, non-volatile, magnetic, optical, or electrical media, such as a random access memory (RAM), read-only memory (ROM), non-volatile RAM (NVRAM), electrically-erasable programmable ROM (EEPROM), ferroelectric RAM (FRAM), flash memory, or any other digital media.

In examples, medical system 100 includes an imaging device 164. Imaging device 164 may enable image guidance, e.g., computed tomography (CT), fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal body portion 110A and therapeutic element 112. For example, a fluoroscopy system (e.g., including a flat-panel detector, x-ray, or c-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic element 112. Further, in some examples, image guidance components (e.g., IVUS, OCT) may be integrated with catheter system 108 (e.g., catheter body 110) and/or run in parallel with catheter system 108 to provide image guidance during positioning of catheter system 108 (e.g., catheter body 110). Imaging device 164 may be configured to communicate with control circuitry 160, catheter system 108, and/or other portions of medical system 100 (e.g., via communication link 165) to receive imaging data, provide image guidance, and/or control, assist in, and/or direct other functions of medical system 100.

In some examples, medical system 100 (e.g., proximal body portion 110B) includes a handle portion 166, which is configured to remain outside vasculature of a patient when distal body portion 110A is within vasculature of the patient. Handle portion 166 may be configured to allow a clinician to navigate at least distal body portion 110A through the vasculature, allow inflation and/or deflation of balloon 114, allow pressurization of therapeutic agent, allow the transmission and/or control of energy delivered to therapeutic element 112, and/or enable other functions of medical system 100 which may assist in the delivery of a treatment (e.g., a neuromodulation) to patient 106. At least some portion of catheter system 108 (e.g., distal body portion 110A) may be substantially flexible, such that catheter system 108 may flex and/or bend enroute to positioning therapeutic element 112 substantially at a target location within a blood vessel of a patient. Hence, although illustrated as substantially linear in FIG. 1, catheter system 108 (or portions thereof) may be configured to assume linear, curved, and/or curvilinear shapes. Correspondingly, longitudinal axis L (and/or portions thereof) defined by catheter system 108 may be linear, curved, and/or curvilinear.

FIG. 2 schematically illustrates a portion of medical system 100 (e.g., distal body portion 110A, balloon 114, therapeutic element 112, and injection element 113) within blood vessel 102 defined by vessel wall 104 of patient 106. Catheter system 108 is configured to enable inflation of balloon 114 (e.g., via inflation fluid flow path 120) to cause catheter system 108 to transition from the delivery configuration (FIG. 1) to an expanded configuration when distal body portion 110A positions balloon 114 and/or therapeutic element 112 within blood vessel 102. In examples, catheter system 108 is configured to expand balloon 114 such that balloon 114 defines a dimension C2 (e.g., a diameter) in an expanded configuration. The dimension C2 defined in the expanded configuration may be greater than the dimension C1 defined in the delivery configuration (FIG. 1). In examples, balloon 114 is configured to define the dimension C1, the dimension C2, and/or another dimension (e.g., a dimension substantially perpendicular to longitudinal axis L) based on an inflation pressure PI of a fluid within balloon interior volume 116.

Catheter system 108 may be configured to control the inflation pressure PI such that the dimension C2 may be adjusted to accommodate the size of blood vessel 102. For example, catheter system 108 may be configured to cause balloon 114 to expand such that balloon 114 (e.g., balloon exterior surface 154) substantially contacts vessel wall 104. Balloon 114 may be configured to position injection element 113 in apposition to, in proximity to, and/or within vessel wall 104 when balloon 114 expands. In examples, catheter system 108 may be configured to substantially center (e.g., center or nearly center to the extent permitted by vessel symmetry) distal body portion 110A within blood vessel 102 when balloon 114 expands to contact vessel wall 104.

Therapeutic element 112 (e.g., injection element 113) is configured to receive a therapeutic agent (e.g., via therapeutic agent flow path 128) and deliver the therapeutic agent to, for example, ablate tissue of vessel wall 104 and/or tissues in proximity to vessel wall 104. Catheter system 108 is configured to pressurize the therapeutic agent (e.g., using therapeutic agent delivery system 126 (FIG. 1)) to cause therapeutic element 112 to receive the therapeutic agent. Catheter system 108 is configured to substantially limit and/or prevent pressurization of the therapeutic agent (e.g., using valve manifold 140 (FIG. 1)) when the inflation pressure PI indicates balloon 114 may not be sufficiently expanded to position therapeutic element 112 and/or injection element 113 against and/or in close proximity to vessel wall 104. Catheter system 108 is configured to permit pressurization of the therapeutic agent (e.g., by a clinician) when the inflation pressure PI indicates balloon 114 has likely inflated and/or remains sufficiently inflated to position therapeutic element 112 and/or injection element 113 against and/or in close proximity to vessel wall 104. Therapeutic element 112 (FIG. 1) may transmit deliver the therapeutic agent to tissue of patient 106 to induce one or more desired effects (e.g., neuromodulation effects) on localized regions of blood vessel 102 and regions adjacent to blood vessel 102. For example, when blood vessel 102 defines a renal artery associated with a kidney 168, therapeutic element 112 may induce one or more desired neuromodulating effects to a portion of Renal Plexus (RP) 170 lying within or adjacent to the adventitia of the renal artery.

FIG. 3 illustrates a portion of medical system 100 positioned within vasculature of a patient 106 to place therapeutic element 112 within blood vessel 102. FIG. 3 is described with primary reference to a renal artery, however similar devices, systems, and techniques may be adapted for accessing other anatomical lumens or tissues within patient 106.

Catheter system 108 (e.g., catheter body 110) may provide access to the renal plexus (RP) through an intravascular path (P), such as a percutaneous access site in the femoral (illustrated), brachial, radial, or axillary artery to a target treatment site within blood vessel 102. By manipulating proximal body portion 110B from outside the intravascular path (P), a clinician may advance at least distal body portion 110A through the sometimes-tortuous intravascular path (P) and remotely manipulate distal body portion 110A. In examples, distal body portion 110A may be remotely manipulated by a clinician using handle portion 166.

In the example illustrated in FIG. 3, balloon 114 is delivered intravascularly to the treatment site using a guidewire 172 in an OTW technique. Catheter system 108 (e.g., catheter body 110) may define a passageway for receiving guidewire 172 for delivery of catheter body 110 (e.g., distal body portion 110A) using either an OTW or a RX technique. At the treatment site, guidewire 172 can be at least partially withdrawn or removed, and balloon 114 may be expanded from the delivery configuration (FIG. 1) to an expanded configuration (FIG. 2). Balloon 114 may substantially position therapeutic element 112 (e.g., injection element 113) relative to blood vessel 102 for delivering energy to blood vessel 102 and/or other anatomical lumens or tissues within patient 106. In other examples, balloon 114 and/or therapeutic element 112 may be delivered to the treatment site within a different guide device, such as guide sheath (not shown in FIG. 3), with or without using guidewire 172. In examples in which medical system 100 includes a guide sheath, when balloon 114 and/or therapeutic element 112 are at the target treatment site, the guide sheath may be at least partially withdrawn or retracted and balloon 114 may be transformed into an expanded configuration. In still other examples, catheter body 110 may be steerable itself such that balloon 114 and/or therapeutic element 112 may be delivered to the treatment site without the aid of guidewire 172 and/or a guide sheath.

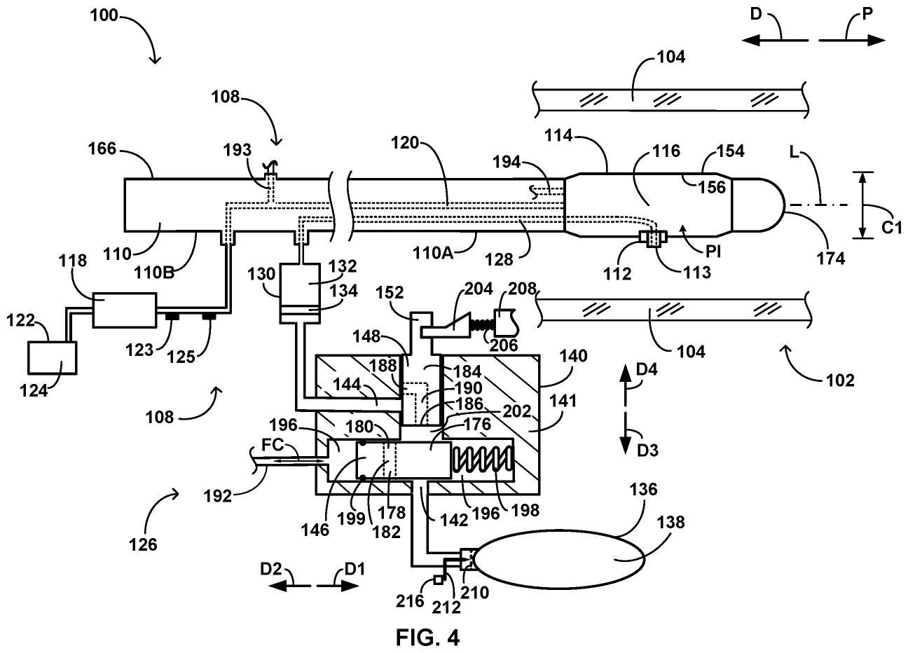

FIG. 4 is a schematic illustration of medical system 100 with catheter system 108 in a delivery configuration within blood vessel 102, with catheter system 108 defining a dimension C1. Valve manifold 140 (e.g., manifold body 141) defines inlet flow path 142 and outlet flow path 144. In FIG. 4, first valve 146 is in the first valve closed position and second valve 148 is in the second valve closed position.

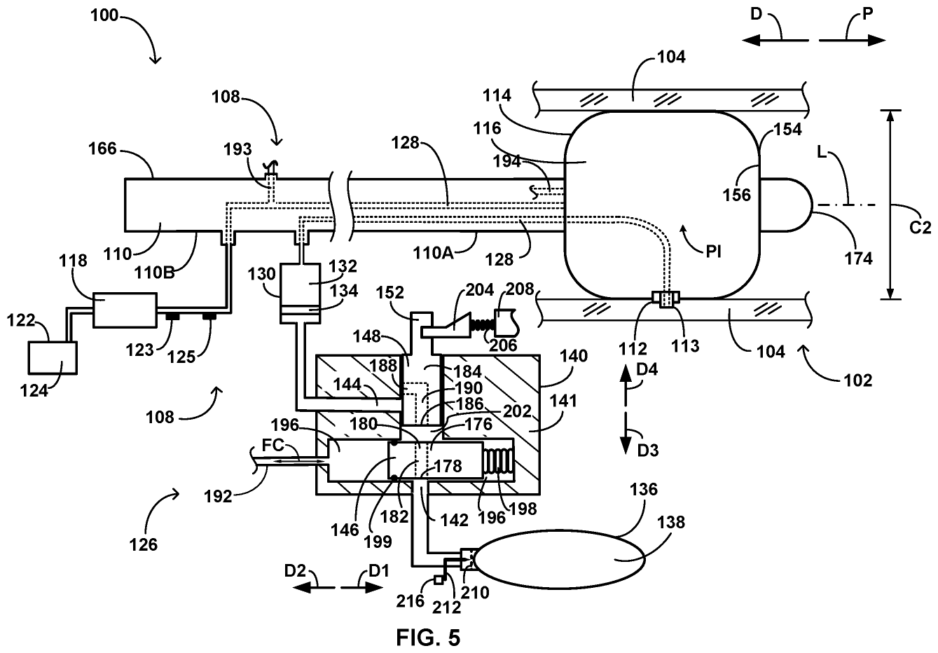

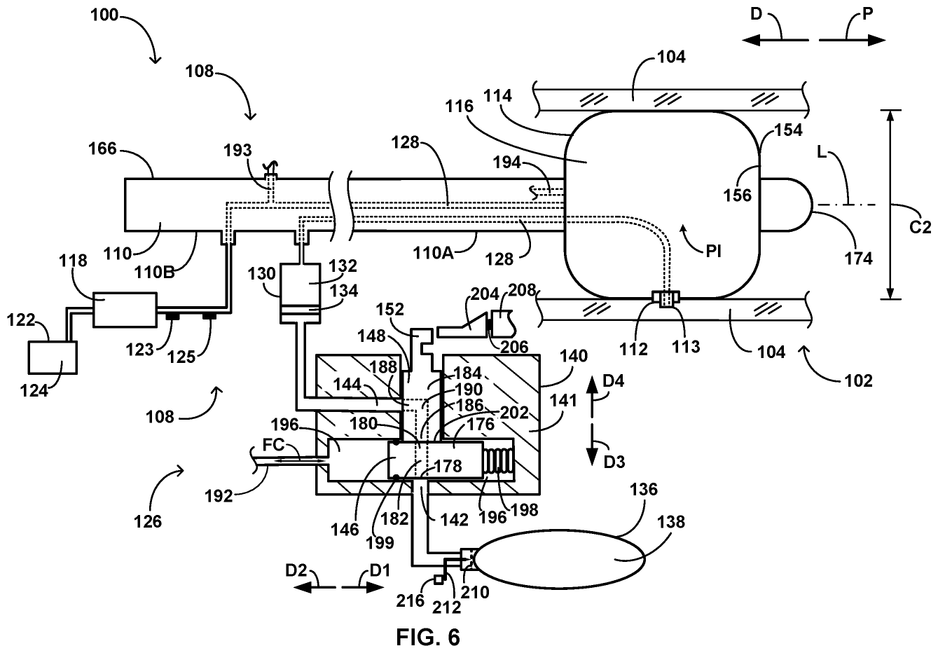

FIG. 5 is a schematic illustration of medical system 100 with catheter system 108 in an expanded configuration within blood vessel 102, with balloon 114 inflated such that catheter system 108 defines a dimension C2 greater than dimension C1. In FIG. 5, first valve 146 is in the first valve open position (e.g., due to the inflation pressure PI within balloon interior volume 116) and second valve 148 is in the second valve closed position. FIG. 6 is a schematic illustration of medical system 100 with catheter system 108 in the expanded configuration within blood vessel 102. In FIG. 6, first valve 146 is in the first valve open position and second valve 148 is in the second valve open position (e.g., due to repositioning of second valve 148 by, e.g., a clinician using actuator 152).

In examples, as shown in FIG. 5 and FIG. 6, catheter system 108 is configured to cause balloon 114 to contact vessel wall 104 in the expanded configuration. Dimension C1 and/or the dimension C2 may be a cross-sectional dimension of catheter system 108 (e.g., balloon 114), the cross-section being taken perpendicular to longitudinal axis L of catheter body 110. In examples, dimension C1 and dimension C2 define a displacement in a direction substantially perpendicular to longitudinal axis L. In examples, longitudinal axis L extends through catheter system 108 through a distal end 174 of catheter body 110 (“body distal end 174”). Longitudinal axis L may extend through at least some portion of distal body portion 110A and/or proximal body portion 110B.

Dimension C1 and/or dimension C2 may define any suitable displacements. Further, catheter system 108 is configured such that dimension C1 and/or dimension C2 may define any one displacement over a range of possible displacements. For example, catheter system 108 may be configured such that balloon 114 and/or catheter body 110 define a minimum cross-sectional dimension (e.g., a minimum diameter) and a maximum cross-sectional dimension (e.g., a maximum diameter) depending on, for example, an inflation pressure PI within interior volume 116. Hence, although illustrated for clarity in FIG. 4, FIG. 5, and FIG. 6 as corresponding to only one displacement value, catheter system 108 is configured such that dimension C1 and dimension C2 may define any displacement greater than or equal to the minimum cross-sectional dimension and less than or equal to the maximum cross-sectional dimension.

Catheter system 108 is configured to generate an inflation pressure PI within balloon interior volume 116 using an inflation fluid provided to balloon interior volume 116 via inflation fluid flow path 120. Interior volume 116 may be bound at least in part by balloon interior surface 156. In examples, inflation fluid flow path 120 is an inflation lumen defined by catheter body 110 (e.g., distal body portion 110A and/or proximal body portion 110B). Balloon 114 is configured to expand radially outward in a direction away from a central longitudinal axis L of catheter body 110 when the inflation pressure PI is sufficient to cause the expansion. Balloon 114 (e.g., balloon body 158) is configured to elastically expand based on the inflation pressure PI within interior volume 116, such that an increase in the inflation pressure PI causes balloon 114 to increase its cross-sectional diameter (e.g., from dimension C1 to dimension C2), and a decrease in the inflation pressure PI causes balloon 114 to decrease its cross-sectional dimension (e.g., from dimension C2 to the dimension C1). Thus, medical system 100 is configured such that catheter system 108 may define a first dimension (e.g., dimension C1) when catheter system 108 is navigated (e.g., by a clinician) to and/or from a target site within blood vessel 102, and define a second dimension greater than the first dimension (e.g., dimension C2) to help hold the therapeutic element 112 in apposition with vessel wall 104, approximately center the catheter within blood vessel 102, and/or assist in other ways.

Catheter system 108 is configured to deliver a therapeutic agent via injection element 113 to a target treatment site, such as a target treatment site on vessel wall 104 or proximate to vessel wall 104 (e.g., within or adjacent to the adventitia). Catheter system 108 may be configured to deliver the therapeutic agent from therapeutic agent container 130 (e.g., therapeutic agent reservoir 132) to injection element 113 using therapeutic agent flow path 128. In examples, at least a portion of therapeutic agent flow path 128 is defined by a lumen defined by catheter body 110. Therapeutic agent container 130 may include pressurization element 134 (e.g., a piston) configured to facilitate the pressurization of the therapeutic agent. In examples, catheter system 108 is configured to cause pressurization element 134 to translate (e.g., translate within therapeutic agent reservoir 132) to pressurize the therapeutic agent within therapeutic agent reservoir 132. Catheter system 108 is configured such that the pressurization of the therapeutic agent within therapeutic agent reservoir 132 drives the therapeutic agent element from therapeutic agent reservoir 132 to injection element 113 via therapeutic agent flow path 128, causing the therapeutic element to discharge from injection element 113.

Catheter system 108 is configured to pressurize the therapeutic element for delivery to injection element 113 using a pressurization fluid. In examples, the pressurization fluid is held in pressurization fluid reservoir 138 of pressurization fluid container 136, which can be, for example a metal and/or polymer canister. Catheter system 108 may be configured to pressurize the therapeutic agent within therapeutic agent reservoir 132 when the pressurized fluid contacts and exerts a pressure on pressurization element 134. Pressurization element 134 may be, for example, a piston, a diaphragm, a bellows, or another component configured to pressurize the therapeutic agent within therapeutic agent reservoir 132 when the pressurization fluid exerts a pressure on pressurization element 134. In examples, pressurization element 134 is a piston configured to translate to pressurize the therapeutic agent within therapeutic agent reservoir 132 when the pressurized fluid contacts and exerts a pressure on the piston.

Catheter system 108 is configured to deliver the pressurization fluid to pressurization element 134 using valve manifold 140. In examples, valve manifold 140 is fluidically in series with pressurization element 134, such that the pressurization fluid flows through a flow path defined by valve manifold 140 in order to reach pressurization element 134. Valve manifold 140 is configured to define the flow path through which the pressurization fluid (e.g., from pressurization fluid reservoir 138) may flow when both first valve 146 is in the first valve open position and second valve 148 is in the second valve open position. Valve manifold 140 is configured to substantially block the flow path (e.g., fluidically isolate pressurization element 134 and pressurization fluid reservoir 138) when first valve 146 is in the first valve closed position and/or second valve 148 is in the second valve closed position.

For example, manifold 140 (e.g., manifold body 141) defines inlet flow path 142 and outlet flow path 144. Manifold 140 is configured to fluidically couple inlet flow path 142 and outlet flow path 144 (e.g., using first valve 146 and second valve 148), such that the pressurization fluid flows through both inlet flow path 142 and outlet flow path 144 when manifold 140 delivers the pressurization fluid to pressurization element 134. First valve 146 is configured to fluidically isolate inlet flow path 142 and outlet flow path 144 when first valve 146 is in the first valve closed position (e.g., as illustrated in FIG. 4). Similarly, second valve 148 is configured to fluidically isolate inlet flow path 142 and outlet flow path 144 when second valve 148 is in the second valve closed position (e.g., as illustrated in FIG. 4). Hence, valve manifold 140 is configured to fluidically couple inlet flow path 142 and outlet flow path 144 when both first valve 146 is in the first valve open position and second valve 148 is in the second valve open position. Valve manifold 140 is configured to fluidically isolate inlet flow path 142 and outlet flow path 144 when either of first valve 146 is in the first valve closed position (regardless of the position of second valve 148) or second valve 148 is in the second valve closed position (regardless of the position of first valve 146).

In examples, first valve 146 includes a valve body 176 (“first valve body 176”) defining a flow path 182 (“first valve flow path 182”). First valve 146 may be configured to fluidically isolate inlet flow path 142 and first valve flow path 182 when first valve 146 is in the first valve closed position (e.g., as depicted in FIG. 4). First valve 146 may be configured to fluidically couple inlet flow path 142 and first valve flow path 182 when first valve 146 is in the first valve open position (e.g., as depicted in FIG. 5 and FIG. 6). In examples, first valve body 176 defines a valve inlet 178 (“first valve inlet 178”) and a valve outlet 180 (“first valve outlet 180”). First valve body 176 may define first valve flow path 182 such that first valve flow path 182 is configured to allow a fluid (e.g., the pressurization fluid) to flow from first valve inlet 178 to first valve outlet 180. In examples, first valve 146 fluidically isolates first valve inlet 178 and inlet flow path 142 (e.g., using a position of manifold body 141) in the first valve closed position (e.g., when first valve 146 fluidically isolates inlet flow path 142 and first valve flow path 182). In examples, first valve 146 fluidically couples first valve inlet 178 and inlet flow path 142 in the first valve open position (e.g., when first valve 146 fluidically couples inlet flow path 142 and first valve flow path 182).

In examples, second valve 148 includes a valve body 184 (“second valve body 184”) defining a flow path 190 (“second valve flow path 190”). Second valve 148 may be configured to fluidically isolate outlet flow path 144 and second valve flow path 190 when second valve 148 is in the second valve closed position (e.g., as depicted in FIG. 4 and FIG. 5). Second valve 148 may be configured to fluidically couple outlet flow path 144 and second valve flow path 190 when second valve 148 is in the second valve open position (e.g., as depicted in FIG. 6). In examples, second valve body 184 defines a valve inlet 186 (“second valve inlet 186”) and a valve outlet 188 (“second valve outlet 188”). Second valve body 184 may define second valve flow path 190 such that second valve flow path 190 is configured to allow a fluid (e.g., the pressurization fluid) to flow from second valve inlet 186 to second valve outlet 188. In examples, second valve 148 fluidically isolates second valve outlet 188 and outlet flow path 144 (e.g., using a position of manifold body 141) in the second valve closed position (e.g., when second valve 148 fluidically isolates second valve flow path 190 and outlet flow path 144). In examples, second valve 148 fluidically couples second valve outlet 188 and outlet flow path 144 in the second valve open position (e.g., when second valve 148 fluidically couples second valve flow path 190 and outlet flow path 144).

Valve manifold 140 is configured to fluidically couple inlet flow path 142 and outlet flow path 144 to deliver the pressurization fluid to pressurization element 134 using first valve flow path 182 and second valve flow path 190. For example, first valve 146 and second valve 148 may be configured such that first valve flow path 182 and second valve flow path 190 are fluidically coupled when both first valve 146 is in the first valve open position and second valve 148 is in the second valve open position (e.g., as illustrated in FIG. 6). Hence, valve manifold 140 is be configured to fluidically couple inlet flow path 142 and outlet flow path 144 to deliver the pressurization fluid to pressurization element 134 when both first valve 146 is in the first valve open position and second valve 148 is in the second valve open position.

In the example shown, first valve 146 and second valve 148 are also configured such that, if either of first valve 146 is in the first valve closed position or second valve 148 is in the second valve closed position, first valve flow path 182 are second valve flow path 190 are fluidically isolated from each other. Hence, valve manifold 140 is be configured to fluidically isolate inlet flow path 142 and outlet flow path 144 to substantially prevent the delivery of pressurization fluid to pressurization element 134 when either of first valve 146 is in the first valve closed position (regardless of the position of second valve 148) or second valve 148 is in the second valve closed position (regardless of the position of first valve 146).

Catheter system 108 is configured to control a position of first valve 146 based on a control pressure, where the control pressure is indicative of (e.g., based on and/or proportional to) the pressure PI within balloon interior volume 116. For example, catheter system 108 may be configured to substantially maintain first valve 146 in the first valve closed position (e.g., fluidically isolating inlet flow path 142 and first valve flow path 182) when the control pressure indicative of the pressure PI is below a threshold pressure. Catheter system 108 may be configured to position first valve 146 in the first valve open position (e.g., fluidically coupling inlet flow path 142 and first valve flow path 182) when the control pressure indicative of the pressure PI is equal to or greater than the pressure threshold. Catheter system 108 may be configured such that a control pressure below the pressure threshold may be indicative that balloon 114 is not sufficiently inflated within vessel 102 (e.g., that balloon 114 may not be inflated sufficiently to position therapeutic element 112 against and/or in close proximity to vessel wall 104). Catheter system 108 may be configured such that a control pressure equal to or greater than the pressure threshold may be indicative that balloon 114 may likely be sufficiently inflated within vessel 102 (e.g., that balloon 114 is likely inflated to position therapeutic element 112 against and/or in close proximity to vessel wall 104).

In examples, valve manifold 140 defines a control fluid flow path 192 configured to receive and/or discharge a control fluid FC. Catheter system 108 may be configured such that control fluid FC exhibits the control pressure indicative of the pressure PI. For example, control fluid flow path 192 may be configured such that the control pressure of control fluid FC is indicative of (e.g., proportional to, equal to, or otherwise based on) a pressure within inflation fluid flow path 120 and/or balloon interior volume 116. In some examples, control fluid control path 192 is fluidically coupled to inflation fluid flow path 120 (e.g., via a flow path 193 defined by catheter body 110) and/or balloon interior volume 116 (e.g., via a flow path 194 defined by catheter body 110). In some examples, control fluid FC and the inflation fluid within inflation fluid path 128 and/or balloon interior volume 116 are portions of a continuous fluid (e.g., a continuous mass of fluid and/or a fluid continuum) extending between control fluid flow path 192 and inflation fluid path 128 and/or balloon interior volume 116. Catheter system 108 may be configured such the inflation pressure PI and/or changes in the inflation pressure PI are transmitted as a fluid pressure to control fluid flow path 192 and/or pressure chamber 150 by the continuous fluid.

Pressure chamber 150 is configured to cause control fluid FC to position first valve 146 based on the inflation pressure PI within balloon interior volume 116. Pressure chamber 150 may be configured such that control fluid FC exerts the control pressure substantially against first valve 146. For examples, pressure chamber 150 may be configured such that the control pressure of control fluid FC acts to exert a force (e.g., an opening force) on first valve 146 tending to position first valve 146 in the first valve open position (e.g., tending to overcome the biasing force and move first valve 146 from the first valve closed position of FIG. 4 to the first valve open position of FIG. 5 and FIG. 6). Hence, valve manifold 140 is configured such that an inflation pressure of the inflation fluid within balloon interior volume 116 may act (e.g., via control fluid FC within pressure chamber 150) on first valve 146 to cause first valve 146 to transition from the first valve closed position to the first valve open position. In examples, pressure chamber 150 is and/or first valve 146 are configured to fluidically isolate control fluid CF and the inflation fluid within valve manifold 140 when control fluid FC exerts the control pressure and/or a force caused by the control pressure against first valve 146.

For example, pressure chamber 150 may be a volume bounded in part by a movable component configured to move relative to valve body 141 when acted upon by the control pressure of control fluid CF. The movable component may be, for example, a portion of first valve 146, a piston, a diaphragm, and/or another component configured to move relative to valve body 141. Valve manifold 140 may be configured such that the movement of the movable component causes first valve 146 to transition from the first valve closed position to the first valve open position. In examples, movement of the movable component causes movement of first valve 146 relative to valve body 141.

In examples, valve manifold 140 includes a biasing element 198 configured to bias first valve 146 in the first valve closed position. Biasing element 198 may be configured to exert a biasing force (e.g., a shutting force) opposing the force exerted on first valve 146 by control fluid FC. In examples, biasing element 198 is configured to exert the biasing force in a direction substantially opposite the force exerted on first valve 146 by control fluid FC. Valve manifold 140 may be configured such that, when the control pressure of control fluid FC within pressure chamber 150 is insufficient to overcome the biasing force of biasing element 198, biasing element 198 causes first valve 146 to remain in the first valve closed position (as illustrated in FIG. 4). Valve manifold 140 may be configured such that, when the control pressure of control fluid FC within pressure chamber 150 is sufficient to overcome the biasing force of biasing element 198 (e.g., when the control pressure is greater than or equal to a pressure threshold), first valve 146 transitions from the first valve open position to the first valve closed position (as illustrated in FIG. 5 and FIG. 6). For example, when the inflation pressure PI within balloon interior volume 116 increases (e.g., when balloon 114 inflates from the dimension C1 to the dimension C2), the control pressure of control fluid FC within pressure chamber 150 may correspondingly increase to overcome the biasing force of biasing element 198, such that first valve 146 transitions from the first valve closed position (e.g., as depicted in FIG. 4) to the first valve open position (e.g., as depicted in FIG. 5 and FIG. 6).

Further, valve manifold 140 may be configured such that, when first valve 146 is in the first valve open position and the control pressure of control fluid FC within pressure chamber 150 decreases (e.g., decreases to below a pressure threshold), biasing element 198 causes first valve 146 to transition from the first valve open position to the first valve closed position. For example, when the inflation pressure PI within balloon interior volume 116 decreases (e.g., when balloon 114 deflates from the dimension C2 to the dimension C1), the control pressure of control fluid FC within pressure chamber 150 may correspondingly decrease such that the biasing force of biasing element 198 overcome the force exerted by the control pressure, causing first valve 146 to transition from the first valve open position to the first valve closed position.

Hence, valve manifold 140 is configured to position first valve 146 based on an inflation pressure PI within balloon interior volume 116. Valve manifold 140 may be configured such that, when the inflation pressure PI causes the control pressure of control fluid FC within pressure chamber 150 to equal or exceed a pressure threshold, valve manifold 140 fluidically couples inlet flow path 142 and first valve flow path 182. Valve manifold 140 may be configured such that, when the inflation pressure PI causes the control pressure of control fluid FC within pressure chamber 150 to be below the pressure threshold, valve manifold 140 fluidically isolates inlet flow path 142 and first valve flow path 182.

Biasing element 198 has any suitable structure configured to bias first valve 146 in the first valve closed position. In examples, biasing element 198 is a compressible element (e.g., a spring). In examples, biasing element 198 is configured to exhibit an extended configuration (as illustrated in FIG. 4) and a compressed condition (as illustrated in FIG. 5 and FIG. 6). Valve manifold 140 may be configured such that first valve 146 is in the first valve closed position when biasing element 198 is in the extended position, and such that first valve 146 is in the first valve open position when biasing element 198 is in the compressed position. In examples, valve manifold 140 is configured such that the position of biasing element 198 is dependent on the control pressure of control fluid FC within pressure chamber 150.

For example, valve manifold 140 may be configured such that, when the control pressure of control fluid FC within pressure chamber 150 is greater than or equal to the threshold pressure, the control pressure causes biasing element 198 to establish the compressed position (e.g., such that valve manifold 140 fluidically couples inlet flow path 142 and first valve flow path 182). Valve manifold 140 may be configured such that, when the control pressure of control fluid FC within pressure chamber 150 is less than or equal to the threshold pressure, the control pressure causes biasing element 198 to establish the extended position (e.g., such that valve manifold 140 fluidically isolates inlet flow path 142 and first valve flow path 182). Hence, in some examples, the threshold pressure causing first valve 146 to transition from the first valve closed position to the first valve open position may be based on an amount of force required to be exerted by control fluid FC (e.g., to cause biasing element 198 to transition from the extended position to the compressed position).

In examples, biasing element 198 is a spring or other compressible element having a spring constant indicative of the force required to cause biasing element 198 to establish the compressed condition. Thus, the pressure threshold of the control pressure causing first valve 146 to transition from the first valve closed position to the first valve open position may be based on (e.g., dependent on and/or proportional to) the spring constant of biasing element 198. Biasing element 198 may include be any device configured to exert a force on first valve 146 which opposed a force exerted on first valve 146 by the control pressure of control fluid FC, such as spring, an electromagnet, or another device. In some examples, valve manifold 140 may be configured such that the force required to cause biasing element 198 to establish the compressed condition may be varied by a clinician to, for example, vary the pressure threshold which causes the first valve 146 to transition from the first valve closed position to the first valve open position. For example, valve manifold 140 may be configured to adjust a position of a spring seat seating biasing element 198, be configured (e.g., using circuitry) to control an electric field of an electromagnet, or be configured to vary the pressure threshold in another manner.

In some examples, first valve 146 is a spool valve. First valve body 176 may define a spool (e.g., a substantially cylindrical spool) configured to translate in a first direction D1 and/or a second direction D2 within a volume 196 (e.g., a spool barrel) defined by valve manifold 140 (e.g., manifold body 141). The spool may define first valve flow path 182 between first valve inlet 178 and first valve outlet 180. In some of these examples, valve manifold 140 is configured such that the pressure of control fluid FC within pressure chamber 150 exerts a force tending to cause the spool to translate in the first direction D1 (e.g., relative to manifold body 141) to cause first valve 146 to transition from the first valve closed position to the first valve open position. Valve manifold 140 may be configured such that biasing element 198 exerts a biasing force tending to cause the spool to translate in the second direction D2 (e.g., relative to manifold body 141) to cause first valve 146 to transition from the first valve open position to the first valve closed position. First valve 146 may be any type of valve configured to position in the first valve closed position and the first valve open position, including a globe valve, a gate valve, a poppet valve, and/or another type of valve.

In some examples, valve manifold 140 and/or first valve 146 includes a resistive element 199 (e.g., an o-ring) configured to reduce a speed (e.g., a speed relative to manifold body 141) at which first valve 146 transitions from the first valve closed position to the first valve open position. Resistive element 199 may be configured to frictionally engage a surface defined by valve manifold 140 (e.g., a surface bounding volume 196) and/or a surface defined by first valve 146 as first valve 146 transitions from the first valve closed position to the first valve open position. The frictional engagement of resistive element 199 may reduce the speed at which first valve 146 transitions when the control pressure of control fluid FC causes first valve 146 to transition from the first valve closed position to the first valve open position. In examples, resistive element 199 is configured to fluidically isolate pressure chamber 150 and one or more other volumes defined by valve manifold 140 (e.g., volume 196 and/or volume 202). For example, resistive element 199 may be configured to limit and/or substantially prevent control fluid FC from flowing from pressure chamber 150 to other volumes defined by valve manifold 140.

Catheter system 108 is configured to control a position of second valve 148 using actuator 152. Actuator 152 is configured to transition second valve 148 between the second valve closed position and the second valve open position. In examples, actuator 152 is configured to transition second valve 148 between the second valve closed position and the second valve open position based on a physical manipulation (e.g., by a clinician) of actuator 152. For example, actuator 152 may include a mechanical element (e.g., a manual valve operator), a button or switch, and/or another component configured to be physically manipulated to cause second valve 148 to transition between the second valve closed position and the second valve open position.

Catheter system 108 is configured such that, when actuator 152 (e.g., due to the action of a clinician) causes second valve 148 to establish the second valve closed position (e.g., as depicted in FIG. 4 and FIG. 5), valve manifold 140 fluidically isolates outlet flow path 144 and second valve flow path 190. Catheter system 108 is configured such that, when second valve 148 is in the second valve closed position, valve manifold 140 fluidically isolates inlet flow path 142 and outlet flow path 144 regardless of the position of first valve 146. Hence, catheter system 108 is configured to prevent a flow of pressurization fluid from pressurization fluid reservoir 138 to pressurization element 134 when second valve 148 is in the second valve closed position regardless of the position of first valve 146. Preventing the flow of pressurization fluid to pressurization element 134 when second valve 148 is in the second valve closed position allows a clinician to control the flow of the pressurization fluid through manifold 140 even when the inflation pressure PI of balloon 114 has caused first valve 146 to position in the first valve open position.

Valve manifold 140 is further configured such that placing second valve 148 in the second valve open position fluidically couples inlet flow path 142 and outlet flow path 144 only when first valve 146 is in the first valve open position. For example, when the pressure PI in balloon interior volume 116 is insufficient to cause first valve 146 to position in the first valve open position, valve manifold 140 continues to fluidically isolate inlet flow path 142 and outlet flow path 144 even when second valve 148 is placed in the second valve open position. Hence, valve manifold 140 is configured such that fluidically coupling inlet flow path 142 and outlet flow path 144 to pressurize the therapeutic agent using pressurization element 134 substantially requires an inflation pressure PI in balloon interior volume 116 sufficient to place first valve 146 in the first valve open position, as well as an action by the clinician to cause actuator 152 to place second valve 148 in the second valve open position. Valve manifold 140 is thus configured to coordinate the inflation pressure PI and the action of the clinician to, for example, assist the clinician in assessing a position of therapeutic element 113 relative to vessel wall 104 prior to causing pressurization of the therapeutic agent using second valve 148. The redundancy provided by valves 146, 148 may help reduce the possibility of inadvertent therapeutic agent delivery to tissue of a patient.

Second valve 148 may be any type of valve configured to position in the second valve closed position and the second valve open position, including a spool valve, a gate valve, a poppet valve, a globe valve, and/or another type of valve. In examples, second valve 148 is a linear motion valve configured to translate in a third direction D3 and/or a fourth direction D4 within a volume 202 defined by valve manifold 140 (e.g., manifold body 141). For example, second valve 148 may be configured to translate in the third direction D3 when second valve 148 transitions from the second valve closed position to the second valve open position, and/or configured to translate in the fourth direction D4 when second valve 148 transitions from the second valve open position to the second valve closed position. In some examples, second valve 148 is a rotary motion valve configured to rotate relative to manifold body 141 within volume 202. For example, second valve 148 may be configured to rotate in a first rotational direction around an axis defined by second valve body 184 when second valve 148 transitions from the second valve closed position to the second valve open position. Second valve 148 may be configured to rotate in a second rotational direction (e.g., opposite the first rotational direction) around the axis defined by second valve body 184 when second valve 148 transitions from the second valve open position to the second valve closed position.

Although the first direction D1 and the second direction D2 (“directions D1, D2”) are illustrated in FIGS. 4-6 as having a specific spatial and/or angular orientation relative to the third direction D3 and the fourth direction D4 (“directions D3, D4), such as substantially perpendicular, this is not required. Directions D1, D2 and directions D3, D4 may have any spatial and/or angular relation to each other. Further, second direction D2 may be a direction substantially opposite first direction D1 or may have another spatial and/or angular orientation relative to first direction D1. Fourth direction D4 may be a direction substantially opposite third direction D3 or may have another spatial and/or angular orientation relative to third direction D3. Any of first direction D1, second direction D2, third direction D3, or fourth direction D4 may have any spatial and/or angular orientation relative to any other of direction D1, second direction D2, third direction D3, or fourth direction D4.

Actuator 152 is configured to exert a force on second valve 148 (e.g., second valve body 184) to cause second valve 148 to transition from the second valve closed position to the second valve open position and/or transition from the second valve open position to the second valve closed position. In examples, actuator 152 is mechanically coupled (e.g., attached) to second valve 148 (e.g., second valve body 184). Actuator 152 may be configured to transmit a linear force and/or rotary force from actuator 152 to second valve body 184. For example, when a clinician exerts a linear force and/or a rotary force on actuator 152, actuator 152 may transmit at least a portion of the linear force and/or rotary force to second valve body 184 to cause second valve 148 to transition between the second valve closed position and the second valve open position.

In examples, actuator 152 is configured to position in a first position when second valve 148 is in the second valve closed position (e.g., as illustrated in FIG. 4 and FIG. 5). Actuator 152 may be configured to position in a second position when second valve 148 is in the second valve open position (e.g., as illustrated in FIG. 6). Actuator 152 may be configured to transition from the first position to the second position (and vice-versa) to cause second valve 148 to transition from the second valve closed position to the second valve open position (and vice-versa). For example, when a clinician manipulates actuator 152 to cause actuator 152 to transition from the first position to the second position (and vice-versa), actuator 152 may cause second valve 148 to transition from the second valve closed position to the second valve open position (and vice-versa). Hence, manifold 140 may be configured such that a clinician may leave and/or position actuator 152 in the first position to prevent and/or limit the fluidic coupling of inlet flow path 142 and outlet flow path 144 (e.g., preventing and/or limiting pressurization of the therapeutic agent within therapeutic agent reservoir 132). Manifold 140 may be configured such that, when first valve 146 is in the first valve open position, a clinician may position and/or leave actuator 152 in the second position to allow the fluidic coupling of inlet flow path 142 and outlet flow path 144 (e.g., causing pressurization of the therapeutic agent within therapeutic agent reservoir 132).

In examples, catheter system 108 includes a switch 204 configured to control the position of actuator 152. Catheter system 108 may be configured such that the fluidic coupling of inlet flow path 142 and outlet flow path 144 (e.g., when first valve 146 is in the first valve open position) requires an initial positioning of switch 204 followed by a positioning of actuator 152. In examples, switch 204 includes a locked position (e.g., as illustrated in FIG. 4 and FIG. 5) and a firing position (e.g., as illustrated in FIG. 6). Catheter system 108 may be configured such that a movement of switch 204 (e.g., caused by a clinician) from the locked position to the firing position must precede a movement of actuator 152 (e.g., caused by a clinician) from the first position to the second position. Hence, catheter system 108 may be configured to prevent the fluidic coupling of inlet flow path 142 and outlet flow path 144 (e.g., preventing and/or limiting pressurization of the therapeutic agent within therapeutic agent reservoir 132) until switch 204 is moved (e.g., by a clinician) from the locked position to the firing position.

In examples, switch 204 is configured such that locked position substantially prevents actuator 152 from transitioning from the first position (which positions second valve 148 in the second valve closed position) to the second position (which positions second valve 148 in the second valve open position). For examples, switch 204 may be configured to limit and/or prevent movement of actuator 152 from transitioning from the first position to the second position. In examples, in the locked position, switch 204 is configured to exert a mechanical force on actuator 152 to limit and/or prevent movement of actuator 152 from transitioning from the first position to the second position. The mechanical force may be a reaction force caused by a force exerted on actuator 152 which causes actuator 152 to exert the force on switch 204. Hence, in the locked position, switch 204 may substantially prevent the fluidic coupling of inlet flow path 142 and outlet flow path 144. Switch 204 may be configured such that the firing position allows (e.g., when positioned by a clinician) actuator 152 to transition from the first position to the second position, such that actuator 152 may cause (e.g., when first valve 146 is in the first valve open position) the fluidic coupling of inlet flow path 142 and outlet flow path 144 (e.g., causing pressurization of the therapeutic agent within therapeutic agent reservoir 132).

In examples, catheter system 108 includes a positioning element 206 configured to bias switch 204 in the locked position. Positioning element 206 may be a compressible element, such as a spring or other compressible element. In examples, positioning element 206 is configured to exert a position biasing force on switch 204 in a direction tending to position switch 204 in the locked position. Switch 204 may be configured such that an opposing force on switch 204 (e.g., exerted by a clinician) which overcomes the position biasing force may cause switch 204 to transition from the locked position to the firing position. Positioning element 206 may be configured to substantially maintain switch 204 in the locked position in the absence of the opposing force on switch 204. In examples, positioning element 206 is mechanically coupled to switch 204 (e.g., at a first end of positioning element 206). Positioning element 206 may be mechanically coupled to a structure 208 (e.g., at a second end of positioning element 206). In examples, structure 208 is configured to remain stationary relative to manifold body 141 during a movement of first valve 146, second valve 148, actuator 152, switch 204, and/or positioning element 206.

In some examples, pressurization fluid container 136 includes a sealing member 210 configured to fluidically isolate pressurization fluid reservoir 138 and inlet flow path 142. Sealing member 210 (e.g., while intact) may be configured to substantially prevent a flow of pressurization fluid from pressurization fluid reservoir 138 to inlet flow path 142. In examples, catheter system 108 includes an actuation member 212 configured to puncture and/or otherwise act on sealing member 210, such that pressurization fluid reservoir 138 is fluidically coupled to inlet flow path 142. Actuation member 212 may be configured such a movement of actuation member 212 (e.g., by a clinician) causes actuation member 212 to puncture and/or otherwise act on sealing member 210 to fluidically couple pressurization fluid reservoir 138 and inlet flow path 142. For examples, actuation member 212 may be configured to move (e.g., relative to sealing member 210 and/or pressurization fluid container 136) from a first actuation member position to a second actuation member position to puncture and/or otherwise act on sealing member 210.

In examples, actuation member 212 includes a piercing nozzle configured to puncture and/or otherwise act on sealing member 210. In some examples, pressurization fluid container 136 is configured to hold a pressurization fluid (e.g., CO₂) at a pressure greater than 500 psi (3447 kPA), such as a pressure of from about 700 psi (4826 kPa) to about 900 psi (6205 kPa) within pressurization fluid reservoir 138. Catheter system 108 may include one or more pressure regulators configured to reduce and/or regulate a pressure of the pressurization fluid. For example, catheter system 108 may include one or more pressure regulators configured to reduce and/or regulate the pressure of the pressurization fluid as the pressurization fluid flows from pressurization fluid reservoir 138 to valve manifold 140.

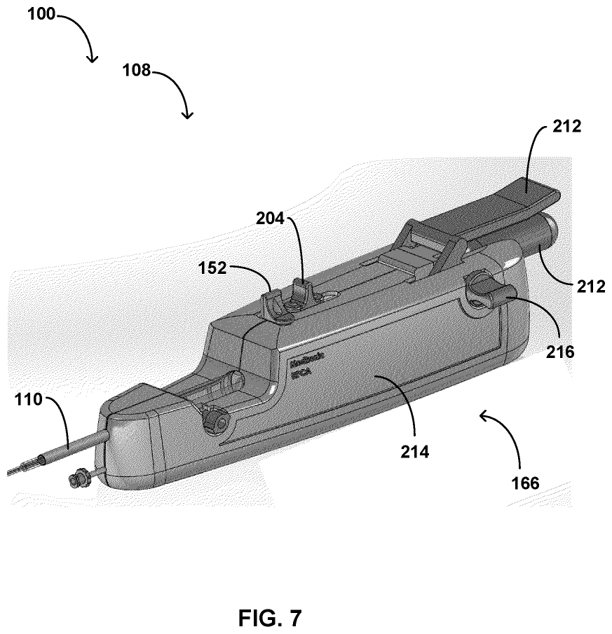

FIG. 7 illustrates a perspective view of a portion of an example catheter system 108, including handle portion 166 and catheter body 110. Handle portion 166 may include a handle housing 214 substantially surrounding and/or mechanically supporting portions of catheter system 108, such as valve manifold 140, pressurization element 134, pressurization fluid container 136, actuator 152, switch 204, actuation member 212, and/or other portions of catheter system 108. In other examples, pressurization fluid container 136 is configured to be mechanically attached to handle 166, e.g., to extend away from handle 166. In examples, catheter system 108 includes a lock 216 (e.g., a lever lock) configured to substantially prevent the movement of actuation member 212 to prevent actuation member 212 from puncturing and/or otherwise acting on sealing member 210 when lock 216 is in a first lock position. In examples, lock 216 is configured to limit and/or prevent actuation member 212 from moving from the first actuation member position to the second actuation member position. Lock 216 may be configured to allow actuation member 212 to puncture and/or otherwise act on sealing member 210 when lock 216 is in a second lock position. Lock 216 may be configured such that clinician may move lock 216 from the first lock position to the second lock position (and vice-versa). In examples, when in the first lock position, lock 216 is configured to exert a force (e.g., a mechanical force) on actuation member 212 to limit and/or prevent movement of actuation member 212 (e.g., from the first actuation member position to the second actuation member position).

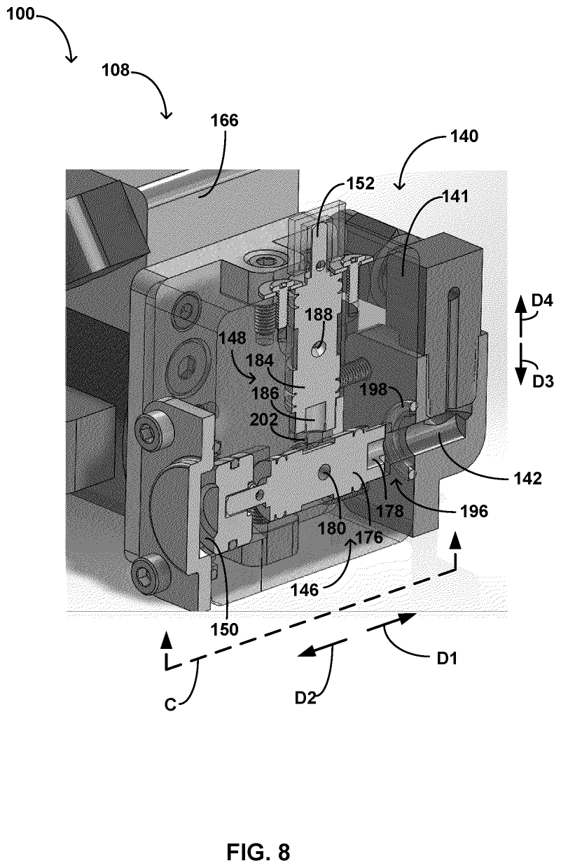

FIG. 8 illustrates a perspective cross-sectional view of an example valve manifold 140, with the cross-section taken along cutting plane C. In FIG. 8, portions of manifold body 141 are illustrated as transparent for clarity. Valve manifold 140 includes biasing element 198 biasing first valve 146 in the first valve closed position, such that first valve outlet 180 is fluidically isolated from second valve inlet 186. Valve manifold 140 is configured to receive a pressurization fluid via inlet flow path 142.

Valve manifold 140 is configured to receive control fluid FC (e.g., a portion of the inflation fluid) in pressure chamber 150. A control pressure of control fluid FC (e.g., caused by inflation pressure PI within balloon interior volume 116) may overcome the biasing force of biasing element 198 and cause translation of first valve 146 in the first direction D1, such that first valve 146 transitions from the first valve closed position to the first valve open position. First valve 146 may fluidically couple (e.g., via first valve inlet 178) inlet flow path 142 and first valve outlet 180 in the first valve open position. In examples, first valve inlet 178 is fluidically coupled to second valve inlet 186 when first valve 146 is in the first valve open position, such that the pressurization fluid may flow from inlet flow path 142 to second valve inlet 186.

Actuator 152 is configured to cause second valve 148 to transition from the second valve closed position to the second valve open position. Second valve 148 fluidically isolates second valve inlet 186 and outlet flow path 144 (FIGS. 1 and 4-6) when second valve 148 is in the second valve closed position. When actuator 152 causes (e.g., due to manipulation of actuator 152 by a clinician) second valve 148 to position in the second valve open position, second valve 148 fluidically couples second valve inlet 186 and the outlet flow path 144, such that the pressurization fluid may flow from inlet flow path 142 to the outlet flow path of manifold 140. Manifold 140 may direct the flow of pressurization fluid to pressurization element 134 to pressurize a therapeutic agent within therapeutic agent reservoir 132 (FIGS. 1 and 4-6).

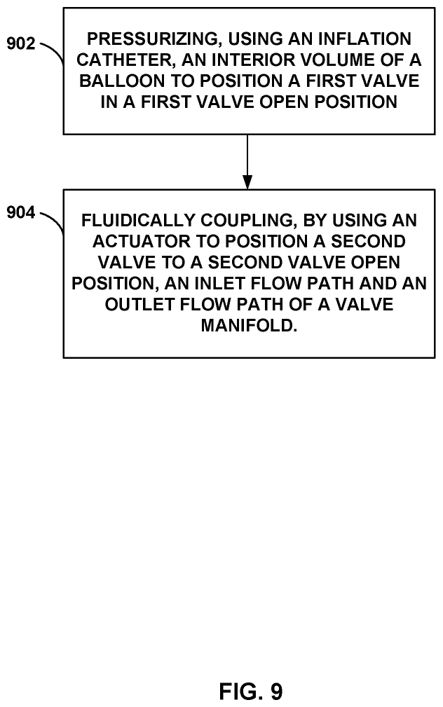

FIG. 9 illustrates a flow diagram of an example technique for pressurizing an interior volume of a balloon. Although the technique is mainly described with reference to system 100 and the components thereof (FIGS. 1-8), the technique may be used with other systems in other examples.

The technique includes pressurizing a balloon interior volume 116 of a balloon 114 using an inflation fluid (902). The inflation fluid may establish an interior pressure PI in balloon interior volume 116. In examples, the interior pressure PI causes a control pressure in a control fluid FC. In examples, control fluid FC is a portion of the inflation fluid. The control pressure may be based on (e.g., dependent on and/or proportional to) the interior pressure PI.

The control pressure of control fluid FC may cause the control fluid to exert a force on a first valve 146 of a valve manifold 140. In examples, the control pressure exerts the force on first valve 146 in a pressure chamber 150 defined by valve manifold 140. The control pressure may cause first valve 146 to move from a first valve closed position to a first valve open position. First valve 146 may include a first valve inlet 178 fluidically coupled to an inlet flow path 142 of valve manifold 140 when first valve 146 is in the first valve open position. In examples, a biasing element 198 exerts a biasing force on first valve 146 tending to position first valve 146 in the first valve closed position. The control pressure of control fluid FC may substantially overcome the biasing force when the control pressure causes first valve 146 to move from the first valve closed position to the first valve open position.

The technique includes fluidically coupling inlet flow path 142 to an outlet flow path 144 of valve manifold by positioning a second valve 148 using an actuator 152 (904). In examples, a clinician positions actuator 152 to position second valve 148 to a second valve open position. In examples, actuator 152 positions from a first position to a second position to transition second valve 148 from a second valve closed position to the second valve open position. In examples, switch 204 moves (e.g., is moved by a clinician) from a locked position to a firing position to allow actuator 152 to transition from the first position to the second position. Second valve 148 may fluidically couple inlet flow path 142 and outlet flow path 144 using second valve flow path 190. Second valve flow path 190 may fluidically couple a second valve inlet 186 defined by a second valve body 184 and a second valve outlet 188 defined by second valve body 184.

In examples, the pressurization fluid causes a pressurization element 134 to pressurize a therapeutic agent held in a therapeutic agent reservoir 132 defined by a therapeutic agent container 130. The therapeutic agent may discharge to an injection element 113 of a therapeutic element 112 when pressurization element 134 pressurizes the therapeutic agent. In examples, balloon 114 positions injection element 113 and/or therapeutic element 112 in contact with and/or in proximity to a vessel wall 104 of a blood vessel 102 of a patient 106 when the inflation fluid establishes interior pressure PI in balloon interior volume 116. In examples, balloon 114 mechanically supports injection element 113 and/or therapeutic element 112 using a balloon exterior surface 154 and/or balloon body 158 when balloon 114 positions injection element 113 and/or therapeutic element 112 in apposition to, in proximity to, and/or within vessel wall 104.

In examples, valve manifold 140 receives the pressurization fluid from a pressurization fluid container 136 defining a pressurization fluid reservoir 138. A sealing member 210 may fluidically isolate pressurization fluid reservoir 138 and inlet flow path 142. An actuation member 212 may move (e.g., be moved by a clinician) to puncture and/or otherwise act on sealing member 210, such that pressurization fluid reservoir 138 is fluidically coupled to inlet flow path 142. In some examples, system 100 reduces and/or regulates a pressure of the pressurization fluid using one or more regulators.

Various examples of the disclosure have been described. Any combination of the described systems, operations, or functions is contemplated. These and other examples are within the scope of the following claims.

Example 1. An medical system, comprising: a first valve biased to a first valve closed position; a valve manifold defining an inlet flow path and an outlet flow path, the valve manifold defining a pressure chamber configured to cause a pressure of a fluid in the pressure chamber to cause the first valve to move from the first valve closed position to a first valve open position; a second valve; and an actuator configured to cause the second valve to position in one of a second valve open position or a second valve closed position, wherein the first valve and the second valve are configured to fluidically couple the inlet flow path and the outlet flow path when the first valve is in the first valve open position and the second valve is in in the second valve open position, and wherein the first valve and the second valve are configured to fluidically isolate the inlet flow path and the outlet flow path when the first valve is in the first valve closed position or the second valve is in the second valve closed position.

Example 2. The medical system of example 1, wherein the first valve defines a first valve outlet fluidically coupled to a first valve inlet, wherein the second valve defines a second valve inlet fluidically coupled to a second valve outlet, and wherein the first valve and the second valve are configured to fluidically couple the first valve outlet and the second valve inlet when the first valve and the second valve fluidically couple the inlet flow path and the outlet flow path.

Example 3. The medical system of example 1 or example 2, wherein the second valve defines a second valve outlet configured to fluidically couple with the outlet flow path when the second valve is in the second valve open position, and wherein the first valve is configured to fluidically couple the inlet flow path and the second valve outlet the first valve is in the first valve open position.

Example 4. The medical system of any of examples 1-3, wherein the fluid is a first fluid, and wherein: the first valve and the second valve define a flow path for a second fluid from the inlet flow path to the outlet flow path when the first valve and the second valve fluidically couple the inlet flow path and the outlet flow path, and the pressure chamber is configured to fluidically isolate the first fluid and the second fluid.

Example 5. The medical system of any of examples 1-4, further comprising a balloon configured to be positioned within a patient, and wherein the pressure chamber is configured such that the pressure of the fluid in the pressure chamber is indicative of a pressure in an interior volume defined by the balloon.

Example 6. The medical system of example 5, wherein the pressure chamber is fluidically coupled to the interior volume defined by the balloon.

Example 7. The medical system of example 5 or example 6, further comprising a catheter defining an inflation lumen configured to deliver an inflation fluid to the interior volume of the balloon to cause the pressure in the interior volume of the balloon.

Example 8. The medical system of any of examples 1-7, further comprising a biasing element configured to bias the first valve to the first valve closed position, wherein the pressure chamber is configured to cause the pressure of the fluid in the pressure chamber to cause the first valve to move from the first valve closed position to the first valve open position when the pressure of the fluid is greater than or equal to a pressure threshold.

Example 9. The medical system of example 8, wherein: the pressure chamber is configured to cause the pressure of the fluid in the pressure chamber to exert an opening force on the first valve to cause the first valve to move from the first valve closed position to the first valve open position, the biasing element is configured to exert a shutting force on the first valve to bias the first valve to the first valve closed position, and the opening force causes the first valve to assume an open position when the opening force overcomes the shutting force.

Example 10. The medical system of any of examples 1-9, further comprising: a container containing a therapeutic agent; a pressurizing element in fluid communication with the container, wherein the second valve is configured to fluidically couple the second valve outlet and the pressurizing element to cause the pressurizing element to pressurize the therapeutic agent when the first valve and the second valve fluidically couple the inlet flow path and the outlet flow path; and a fluid delivery tube configured to deliver the therapeutic agent to a target site within a patient when the pressurizing element pressurizes the therapeutic agent.

Example 11. The medical system of example 10, wherein the second valve is configured to fluidically couple the second valve outlet and the pressurizing element when the second valve is positioned in the second valve open position, and wherein the second valve is configured to fluidically isolate the second valve outlet and the pressurizing element when the second valve is positioned in the second valve closed position.

Example 12. The medical system of any of examples 1-11, wherein the actuator is configured to position in a first position to cause the second valve to position in the second valve closed position, and wherein the actuator is configured to position in a second position to cause the second valve to position in the second valve open position, and further comprising a switch configured to establish a locked position and a firing position, wherein the switch is configured to: prevent the actuator from transitioning from the first position to the second position when the switch is in the locked position, and allow the actuator to transition from the first position to the second position when the switch is in the firing position.

Example 13. The medical system of example 12, further comprising a positioning element configured to exert a first force on the switch, wherein the first force tends to cause the switch to establish the locked position, and wherein the switch is configured to establish the firing position when a second force on the switch overcomes the first force.

Example 14. The medical system of any of example 1-13, further comprising: a fluid container defining a reservoir, wherein the fluid container includes a sealing member configured to fluidically isolate the reservoir from the first valve inlet; and an actuation member configured to position to puncture the sealing member to fluidically couple the reservoir and the first valve inlet.

Example 15. The medical system of any of examples 1-14, wherein the first valve includes a valve spool configured to translate in a first direction to cause the first valve to assume the first valve closed position and configured to translate in a second direction substantially opposite the first direction to assume the first valve open position.

Example 16. The medical system of example 15, further comprising a biasing element configured to bias the first valve to the first valve closed position, wherein the biasing element is configured to cause the valve spool to translate in a second direction substantially opposite the first direction.

Example 17. The medical system of any of examples 1-16, further comprising a piston, wherein the pressure chamber is configured to cause the pressure of the fluid to translate the piston, and wherein the piston is configured to exert an opening force on the first valve when the piston translates to cause the first valve to move from the first valve closed position to the first valve open position.

Example 18. The medical system of example 17, further comprising a resistive element positioned on a periphery of the piston, wherein the resistive element is configured to reduce a speed of the translation of the piston.

Example 19. The medical system of examples 1-18, further comprising a diaphragm, wherein the pressure chamber is configured to cause the pressure of the fluid to deflect the diaphragm, and wherein the diaphragm is configured to exert an opening force on the first valve when the diaphragm deflects to cause the first valve to move from the first valve closed position to a first valve open position.

Example 20. A method, comprising: pressurizing, using an inflation catheter, an interior volume of a balloon using an inflation fluid, wherein the inflation fluid is configured to establish a pressure in a pressure chamber of a valve manifold, the pressure being sufficient to cause a first valve to move from a first valve closed position to a first valve open position, the first valve being biased to the first valve closed position; and fluidically coupling an inlet flow path of a valve manifold to an outlet flow path of the valve manifold by at least transitioning, using an actuator, a second valve from a second valve closed position to a second valve open position, wherein the first valve and the second valve are configured to fluidically couple the inlet flow path and the outlet flow path when the first valve is in the first valve open position and the second valve is in the second valve open position, and wherein the first valve and the second valve are configured to fluidically isolate the inlet flow path and the outlet flow path valve when at least one the first valve is in the first valve closed position or second valve is in the second valve closed position.

Example 21. The method of claim 20, further comprising: transitioning a switch from a locked position to a firing position; and positioning the actuator from a first position to a second position when the switch is transitioned to the firing position, wherein the actuator is configured to cause the second valve to position in the second valve closed position in the first position and configured to position the second valve in the second valve open in the second position, and wherein the switch is configured to prevent the actuator from transitioning from the first position to the second position when the switch is in the locked position and configured to allow the actuator to transition from the first position to the second position when the switch is in the firing position.

Example 22. The method of example 21, further comprising exerting, using a positioning element, a first force on the switch, wherein the first force causes the switch to establish the locked position.

Example 23. The method of any of examples 20-22, wherein when the inlet flow path and the outlet flow path fluidically couple, a first flow path defined by a first valve body of the first valve and a second flow path defined by a second valve body of the second valve are fluidically coupled.

Example 24. The method of any of example 20-23, wherein the first valve and the second valve define a flow path for a second fluid when the inlet flow path and the outlet flow path are fluidically coupled; and wherein the fluid in the pressure chamber is fluidically isolated from the second fluid.

Example 25. The method of any of examples 20-24, further comprising exerting a shutting force, using a biasing element, on the first valve to bias the first valve in the first valve closed position.

Example 26. The method of any of examples 20-25, further comprising positioning, using the bias of the first valve, the first valve in the first valve closed position when the pressure of the fluid in the pressure chamber decreases below a pressure threshold.

Example 27. The method of any of claims 20-26, further comprising: pressurizing, using a pressurizing element in fluid communication with a container, a therapeutic agent in the container when the first valve and the second valve fluidically couple the inlet flow path and the outlet flow path; and delivering, using a fluid delivery tube, the pressurized therapeutic agent to a target site within a patient.

Example 28. The method of claim 27, wherein: a second valve outlet of the second valve and the pressurizing element are fluidically coupled when the valve actuator positions the second valve in the second valve open position; or the second valve outlet and the pressurizing element are fluidically isolated when the valve actuator positions the second valve in the second valve closed position.

Example 29. The medical system of any of examples 20-28, further comprising puncturing, using an actuation member, a sealing member of a fluid container defining a reservoir to fluidically couple the reservoir and the valve inlet.

Further disclosed herein is the subject-matter of the following clauses:
1. A medical system, comprising:
   a first valve biased to a first valve closed position;
   a valve manifold defining an inlet flow path and an outlet flow path, the valve manifold defining a pressure chamber configured to cause a pressure of a fluid in the pressure chamber to cause the first valve to move from the first valve closed position to a first valve open position;
   a second valve; and
   an actuator configured to cause the second valve to position in one of a second valve open position or a second valve closed position,
      wherein the first valve and the second valve are configured to fluidically couple the inlet flow path and the outlet flow path when the first valve is in the first valve open position and the second valve is in in the second valve open position, and
      wherein the first valve and the second valve are configured to fluidically isolate the inlet flow path and the outlet flow path when the first valve is in the first valve closed position or the second valve is in the second valve closed position.
2. The medical system of clause 1,
   wherein the first valve defines a first valve outlet fluidically coupled to a first valve inlet,
   wherein the second valve defines a second valve inlet fluidically coupled to a second valve outlet, and
   wherein the first valve and the second valve are configured to fluidically couple the first valve outlet and the second valve inlet when the first valve and the second valve fluidically couple the inlet flow path and the outlet flow path.
3. The medical system of clause 1 or clause 2,
   wherein the second valve defines a second valve outlet configured to fluidically couple with the outlet flow path when the second valve is in the second valve open position, and
   wherein the first valve is configured to fluidically couple the inlet flow path and the second valve outlet the first valve is in the first valve open position.
4. The medical system of any of clauses 1-3, wherein the fluid is a first fluid, and wherein:
   the first valve and the second valve define a flow path for a second fluid from the inlet flow path to the outlet flow path when the first valve and the second valve fluidically couple the inlet flow path and the outlet flow path, and
   the pressure chamber is configured to fluidically isolate the first fluid and the second fluid.
5. The medical system of any of clauses 1-4, further comprising a balloon configured to be positioned within a patient, and wherein the pressure chamber is configured such that the pressure of the fluid in the pressure chamber is indicative of a pressure in an interior volume defined by the balloon.
6. The medical system of clause 5, wherein the pressure chamber is fluidically coupled to the interior volume defined by the balloon.
7. The medical system of clause 5 or clause 6, further comprising a catheter defining an inflation lumen configured to deliver an inflation fluid to the interior volume of the balloon to cause the pressure in the interior volume of the balloon.
8. The medical system of any of clauses 1-7, further comprising a biasing element configured to bias the first valve to the first valve closed position, wherein the pressure chamber is configured to cause the pressure of the fluid in the pressure chamber to cause the first valve to move from the first valve closed position to the first valve open position when the pressure of the fluid is greater than or equal to a pressure threshold.
9. The medical system of clause 8, wherein:
   the pressure chamber is configured to cause the pressure of the fluid in the pressure chamber to exert an opening force on the first valve to cause the first valve to move from the first valve closed position to the first valve open position,
   the biasing element is configured to exert a shutting force on the first valve to bias the first valve to the first valve closed position, and
   the opening force causes the first valve to assume an open position when the opening force overcomes the shutting force.
10. The medical system of any of clauses 1-9, further comprising:
   a container containing a therapeutic agent;
   a pressurizing element in fluid communication with the container, wherein the second valve is configured to fluidically couple the second valve outlet and the pressurizing element to cause the pressurizing element to pressurize the therapeutic agent when the first valve and the second valve fluidically couple the inlet flow path and the outlet flow path; and
   a fluid delivery tube configured to deliver the therapeutic agent to a target site within a patient when the pressurizing element pressurizes the therapeutic agent.
11. The medical system of clause 10,
   wherein the second valve is configured to fluidically couple the second valve outlet and the pressurizing element when the second valve is positioned in the second valve open position, and
   wherein the second valve is configured to fluidically isolate the second valve outlet and the pressurizing element when the second valve is positioned in the second valve closed position.
12. The medical system of any of clauses 1-11, wherein the actuator is configured to position in a first position to cause the second valve to position in the second valve closed position, and wherein the actuator is configured to position in a second position to cause the second valve to position in the second valve open position, and further comprising a switch configured to establish a locked position and a firing position, wherein the switch is configured to:
   prevent the actuator from transitioning from the first position to the second position when the switch is in the locked position, and
   allow the actuator to transition from the first position to the second position when the switch is in the firing position.
13. The medical system of clause 12, further comprising a positioning element configured to exert a first force on the switch, wherein the first force tends to cause the switch to establish the locked position, and wherein the switch is configured to establish the firing position when a second force on the switch overcomes the first force.
14. The medical system of any of clauses 1-13, further comprising:
   a fluid container defining a reservoir, wherein the fluid container includes a sealing member configured to fluidically isolate the reservoir from the first valve inlet; and
   an actuation member configured to position to puncture the sealing member to fluidically couple the reservoir and the first valve inlet.
15. The medical system of any of clauses 1-14, wherein the first valve includes a valve spool configured to translate in a first direction to cause the first valve to assume the first valve closed position and configured to translate in a second direction substantially opposite the first direction to assume the first valve open position.
16. The medical system of clause 15, further comprising a biasing element configured to bias the first valve to the first valve closed position, wherein the biasing element is configured to cause the valve spool to translate in a second direction substantially opposite the first direction.
17. The medical system of any of clauses 1-16, further comprising a piston, wherein the pressure chamber is configured to cause the pressure of the fluid to translate the piston, and wherein the piston is configured to exert an opening force on the first valve when the piston translates to cause the first valve to move from the first valve closed position to the first valve open position.
18. The medical system of clause 17, further comprising a resistive element positioned on a periphery of the piston, wherein the resistive element is configured to reduce a speed of the translation of the piston.
19. The medical system of clauses 1-18, further comprising a diaphragm, wherein the pressure chamber is configured to cause the pressure of the fluid to deflect the diaphragm, and wherein the diaphragm is configured to exert an opening force on the first valve when the diaphragm deflects to cause the first valve to move from the first valve closed position to a first valve open position.
20. a Method, Comprising:
   pressurizing, using an inflation catheter, an interior volume of a balloon using an inflation fluid, wherein the inflation fluid is configured to establish a pressure in a pressure chamber of a valve manifold, the pressure being sufficient to cause a first valve to move from a first valve closed position to a first valve open position, the first valve being biased to the first valve closed position; and
   fluidically coupling an inlet flow path of a valve manifold to an outlet flow path of the valve manifold by at least transitioning, using an actuator, a second valve from a second valve closed position to a second valve open position,
   wherein the first valve and the second valve are configured to fluidically couple the inlet flow path and the outlet flow path when the first valve is in the first valve open position and the second valve is in the second valve open position, and
   wherein the first valve and the second valve are configured to fluidically isolate the inlet flow path and the outlet flow path valve when at least one the first valve is in the first valve closed position or second valve is in the second valve closed position.
21. The method of clause 20, further comprising:
   transitioning a switch from a locked position to a firing position; and
   positioning the actuator from a first position to a second position when the switch is transitioned to the firing position,
   wherein the actuator is configured to cause the second valve to position in the second valve closed position in the first position and configured to position the second valve in the second valve open in the second position, and
   wherein the switch is configured to prevent the actuator from transitioning from the first position to the second position when the switch is in the locked position and configured to allow the actuator to transition from the first position to the second position when the switch is in the firing position.
22. The method of clause 21, further comprising exerting, using a positioning element, a first force on the switch, wherein the first force causes the switch to establish the locked position.
23. The method of any of clauses 20-22, wherein when the inlet flow path and the outlet flow path fluidically couple, a first flow path defined by a first valve body of the first valve and a second flow path defined by a second valve body of the second valve are fluidically coupled.
24. The method of any of clauses 20-23,
   wherein the first valve and the second valve define a flow path for a second fluid when the inlet flow path and the outlet flow path are fluidically coupled; and
   wherein the fluid in the pressure chamber is fluidically isolated from the second fluid.
25. The method of any of clauses 20-24, further comprising exerting a shutting force, using a biasing element, on the first valve to bias the first valve in the first valve closed position.
26. The method of any of clauses 20-25, further comprising positioning, using the bias of the first valve, the first valve in the first valve closed position when the pressure of the fluid in the pressure chamber decreases below a pressure threshold.
27. The method of any of clauses 20-26, further comprising:
   pressurizing, using a pressurizing element in fluid communication with a container, a therapeutic agent in the container when the first valve and the second valve fluidically couple the inlet flow path and the outlet flow path; and
   delivering, using a fluid delivery tube, the pressurized therapeutic agent to a target site within a patient.
28. The method of clause 27, wherein:
   a second valve outlet of the second valve and the pressurizing element are fluidically coupled when the valve actuator positions the second valve in the second valve open position; or
   the second valve outlet and the pressurizing element are fluidically isolated when the valve actuator positions the second valve in the second valve closed position.
29. The medical system of any of clauses 20-28, further comprising puncturing, using an actuation member, a sealing member of a fluid container defining a reservoir to fluidically couple the reservoir and the valve inlet.

1: A medical system, comprising:
a first valve biased to a first valve closed position;
a valve manifold defining an inlet flow path and an outlet flow path, the valve manifold defining a pressure chamber configured to cause a pressure of a fluid in the pressure chamber to cause the first valve to move from the first valve closed position to a first valve open position;
a second valve; and
an actuator configured to cause the second valve to position in one of a second valve open position or a second valve closed position,
wherein the first valve and the second valve are configured to fluidically couple the inlet flow path and the outlet flow path when the first valve is in the first valve open position and the second valve is in in the second valve open position, and
wherein the first valve and the second valve are configured to fluidically isolate the inlet flow path and the outlet flow path when the first valve is in the first valve closed position or the second valve is in the second valve closed position.

2: The medical system of claim 1,
wherein the first valve defines a first valve outlet fluidically coupled to a first valve inlet,
wherein the second valve defines a second valve inlet fluidically coupled to a second valve outlet, and
wherein the first valve and the second valve are configured to fluidically couple the first valve outlet and the second valve inlet when the first valve and the second valve fluidically couple the inlet flow path and the outlet flow path.

3: The medical system of claim 1,
wherein the second valve defines a second valve outlet configured to fluidically couple with the outlet flow path when the second valve is in the second valve open position, and
wherein the first valve is configured to fluidically couple the inlet flow path and the second valve outlet the first valve is in the first valve open position.

4: The medical system of claim 1, wherein the fluid is a first fluid, and wherein:
the first valve and the second valve define a flow path for a second fluid from the inlet flow path to the outlet flow path when the first valve and the second valve fluidically couple the inlet flow path and the outlet flow path, and
the pressure chamber is configured to fluidically isolate the first fluid and the second fluid.

5: The medical system of claim 1, further comprising a balloon configured to be positioned within a patient, and wherein the pressure chamber is configured such that the pressure of the fluid in the pressure chamber is indicative of a pressure in an interior volume defined by the balloon.

6: The medical system of claim 5, wherein the pressure chamber is fluidically coupled to the interior volume defined by the balloon.

7: The medical system of claim 5, further comprising a catheter defining an inflation lumen configured to deliver an inflation fluid to the interior volume of the balloon to cause the pressure in the interior volume of the balloon.

8: The medical system of claim 1, further comprising a biasing element configured to bias the first valve to the first valve closed position, wherein the pressure chamber is configured to cause the pressure of the fluid in the pressure chamber to cause the first valve to move from the first valve closed position to the first valve open position when the pressure of the fluid is greater than or equal to a pressure threshold.

9: The medical system of claim 8, wherein:
the pressure chamber is configured to cause the pressure of the fluid in the pressure chamber to exert an opening force on the first valve to cause the first valve to move from the first valve closed position to the first valve open position,
the biasing element is configured to exert a shutting force on the first valve to bias the first valve to the first valve closed position, and
the opening force causes the first valve to assume an open position when the opening force overcomes the shutting force.

10: The medical system of claim 1, further comprising:
a container containing a therapeutic agent;
a pressurizing element in fluid communication with the container, wherein the second valve is configured to fluidically couple the second valve outlet and the pressurizing element to cause the pressurizing element to pressurize the therapeutic agent when the first valve and the second valve fluidically couple the inlet flow path and the outlet flow path; and
a fluid delivery tube configured to deliver the therapeutic agent to a target site within a patient when the pressurizing element pressurizes the therapeutic agent.

11: The medical system of claim 10,
wherein the second valve is configured to fluidically couple the second valve outlet and the pressurizing element when the second valve is positioned in the second valve open position, and
wherein the second valve is configured to fluidically isolate the second valve outlet and the pressurizing element when the second valve is positioned in the second valve closed position.

12: The medical system of claim 1, wherein the actuator is configured to position in a first position to cause the second valve to position in the second valve closed position, and wherein the actuator is configured to position in a second position to cause the second valve to position in the second valve open position, and further comprising a switch configured to establish a locked position and a firing position, wherein the switch is configured to:
prevent the actuator from transitioning from the first position to the second position when the switch is in the locked position, and
allow the actuator to transition from the first position to the second position when the switch is in the firing position.

13: The medical system of claim 12, further comprising a positioning element configured to exert a first force on the switch, wherein the first force tends to cause the switch to establish the locked position, and wherein the switch is configured to establish the firing position when a second force on the switch overcomes the first force.

14: The medical system of claim 1, further comprising:
a fluid container defining a reservoir, wherein the fluid container includes a sealing member configured to fluidically isolate the reservoir from the first valve inlet; and
an actuation member configured to position to puncture the sealing member to fluidically couple the reservoir and the first valve inlet.

15: The medical system of claim 1, wherein the first valve includes a valve spool configured to translate in a first direction to cause the first valve to assume the first valve closed position and configured to translate in a second direction substantially opposite the first direction to assume the first valve open position.

16: The medical system of claim 15, further comprising a biasing element configured to bias the first valve to the first valve closed position, wherein the biasing element is configured to cause the valve spool to translate in a second direction substantially opposite the first direction.

17: The medical system of claim 1, further comprising a piston, wherein the pressure chamber is configured to cause the pressure of the fluid to translate the piston, and wherein the piston is configured to exert an opening force on the first valve when the piston translates to cause the first valve to move from the first valve closed position to the first valve open position.

18: The medical system of claim 17, further comprising a resistive element positioned on a periphery of the piston, wherein the resistive element is configured to reduce a speed of the translation of the piston.

19: The medical system of claim 1, further comprising a diaphragm, wherein the pressure chamber is configured to cause the pressure of the fluid to deflect the diaphragm, and wherein the diaphragm is configured to exert an opening force on the first valve when the diaphragm deflects to cause the first valve to move from the first valve closed position to a first valve open position.

20: A method, comprising:
pressurizing, using an inflation catheter, an interior volume of a balloon using an inflation fluid, wherein the inflation fluid is configured to establish a pressure in a pressure chamber of a valve manifold, the pressure being sufficient to cause a first valve to move from a first valve closed position to a first valve open position, the first valve being biased to the first valve closed position; and
fluidically coupling an inlet flow path of a valve manifold to an outlet flow path of the valve manifold by at least transitioning, using an actuator, a second valve from a second valve closed position to a second valve open position,
wherein the first valve and the second valve are configured to fluidically couple the inlet flow path and the outlet flow path when the first valve is in the first valve open position and the second valve is in the second valve open position, and
wherein the first valve and the second valve are configured to fluidically isolate the inlet flow path and the outlet flow path valve when at least one the first valve is in the first valve closed position or second valve is in the second valve closed position.
